(19) United States
(12) Patent Application Publication
(10) Pub. No.: US 2006/0141049 A1
(43) Pub. Date: Jun. 29, 2006

(54) Triamcinolone compositions for intravitreal administration to treat ocular conditions

(75) Inventors: Robert T. Lyons, Laguna Hills, CA (US); James N. Chang, Newport Beach, CA (US); John T. Trogden, Anaheim, CA (US); Scott M. Whitcup, Laguna Hills, CA (US)

Correspondence Address: ALLERGAN, INC., LEGAL DEPARTMENT, 2525 DUPONT DRIVE, T2-7H, IRVINE, CA, 92612-1599, US

(73) Assignee: ALLERGAN, INC., Irvine, CA (US)

(21) Appl. No.: 11/354,415

(22) Filed: Feb. 14, 2006

Related U.S. Application Data
(63) Continuation-in-part of application No. 10/966,764, filed on Oct. 14, 2004.
(60) Provisional application No. 60/530,062, filed on Dec. 16, 2003.
(60) Provisional application No. 60/519,237, filed on Nov. 12, 2003.

(51) Int. Cl. A61K 31/728; A61K 31/695; A61K 9/14

(52) U.S. Cl. 424/489; 514/54; 514/63

(57) ABSTRACT

Triamcinolone compositions, and methods of using such compositions, useful for injection into the vitreous of human eyes are provided. Such compositions can include triamcinolone particles present in a therapeutically effective amount, a viscosity inducing component, and an aqueous carrier component. The compositions have viscosities of at least about 10 cps or about 100 cps at a shear rate of 0.1/second. In a preferred embodiment, the viscosity is in the range of from about 80,000 cps to about 300,000 cps. The compositions advantageously suspend the triamcinolone particles for prolonged periods of time.

### CROSS REFERENCE

This application is a continuation in part of application Ser. No. 10/966,764, filed Oct. 14, 2004, which application claims the benefit of provisional patent application Ser. No. 60/519,237, filed Nov. 12, 2003 and provisional patent application Ser. No. 60/530,062, filed Dec. 16, 2003, all of which applications are hereby incorporated herein by reference in their entireties.

### BACKGROUND

The present invention relates to triamcinolone compositions and methods for treating and/or preventing ocular conditions, such as anterior ocular conditions and posterior ocular conditions. In particular the present invention relates to extended release and sustained release triamcinolone compositions, including injectable implants, for treating posterior ocular conditions.

A pharmaceutical composition (synonymously a composition) is a formulation which contains at least one active ingredient (for example a corticosteroid such as a triamcinolone) as well as, for example, one or more excipients, buffers, carriers, stabilizers, preservatives and/or bulking agents, and is suitable for administration to a patient to achieve a desired effect or result. The pharmaceutical compositions disclosed herein can have diagnostic, therapeutic, cosmetic and/or research utility in various species, such as for example in human patients or subjects.

An ocular condition can include a disease, aliment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball. An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves, the conjunctiva, the cornea, the conjunctiva, the anterior chamber, the iris, the posterior chamber (behind the retina but in front of the posterior wall of the lens capsule), the lens or the lens capsule and blood vessels and nerve which vascularize or innervate an anterior ocular region or site. A posterior ocular (also referred to herein synonymously as a “posterior segment”) condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular (or posterior segment) region or site.

Thus, a posterior ocular condition can include a disease, ailment or condition, such as for example, macular degeneration (such as non-exudative age related macular degeneration and exudative age related macular degeneration); choroidal neovascularization; acute macular neuroretinopathy; macular edema (such as cystoid macular edema and diabetic macular edema); Behcet's disease, retinal disorders, diabetic retinopathy (including proliferative diabetic retinopathy); retinal arterial occlusive disease; central retinal vein occlusion; uveitis (including intermediate and anterior uveitis); retinal detachment; ocular trauma which affects a posterior ocular site or location; a posterior ocular condition caused by or influenced by an ocular laser treatment; posterior ocular conditions caused by or influenced by a photodynamic therapy; photocoagulation; radiation retinopathy; epiretinal membrane disorders; branch retinal vein occlusion; anterior ischemic optic neuropathy; non-retinopathy diabetic retinal dysfunction, retinitis pigmentosa and glaucoma. Glaucoma can be considered a posterior ocular condition because a therapeutic goal can be to prevent the loss of or reduce the occurrence of loss of vision due to damage to or loss of retinal cells or optic nerve cells (i.e. neuroprotection).

An anterior ocular condition can include a disease, ailment or condition, such as for example, aphakia; pseudophakia; astigmatism; blepharospasm; cataract; conjunctival diseases; conjunctivitis; corneal diseases;, corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Macular edema is a major cause of visual loss in patients with diabetes, central retinal vein occlusion (CRVO) and branch retinal vein occlusion (BRVO). Although laser photocoagulation can reduce further vision loss in patients with diabetic macular edema (DME), vision that has already been decreased by macular edema usually does not improve by use of laser photocoagulation. Currently, there is no FDA (U.S. Food and Drug Administration) approved treatment for macular edema associated with CRVO. For macular edema associated with BRVO, grid laser photocoagulation may be an effective treatment for some patients.

Diabetic macular edema results from abnormal leakage of macromolecules, such as lipoproteins, from retinal capillaries into the extravascular space followed by an oncotic influx of water into the extravascular space. Abnormalities in the retinal pigment epithelium may also cause or contribute to diabetic macular edema. These abnormalities can allow increased fluid from the choriocapillaries to enter the retina or they may decrease the normal efflux of fluid from the retina to the choriocapillaries. The mechanism of breakdown of the blood-retina barrier at the level of the retinal capillaries and the retinal pigment epithelium may be due to changes to tight junction proteins such as occludin. Antcliff R., et al Marshall J., The pathogenesis of edema in diabetic maculopathy, Semin Ophthalmol 1999; 14:223-232.

Macular edema from venous occlusive disease can result from thrombus formation at the lamina cribrosa or at an arteriovenous crossing. These changes can result in an increase in retinal capillary permeability and accompanying retinal edema. The increase in retinal capillary permeability and subsequent retinal edema can ensue from of a breakdown of the blood retina barrier mediated in part by vascular endothelial growth factor (VEGF), a 45 kD glycoprotein, as it is known that VEGF can increase vascular permeability. VEGF may regulate vessel permeability by increasing phosphorylation of tight junction proteins such as occludin and zonula occluden. Similarly, in human non-ocular disease states such as ascites, VEGF has been characterized as a potent vascular permeability factor (VPF).

The normal human retina contains little or no VEGF; however, hypoxia causes upregulation of VEGF production. Disease states characterized by hypoxia-induced VEGF upregulation include CRVO and BRVO. This hypoxia induced upregulation of VEGF can be inhibited pharmacologically. Pe'er J. et al., Vascular endothelial growth factor upregulation in human central retinal vein occlusion, Ophthalmology 1998; 105:412-416. It has been demonstrated that anti-VEGF antibodies can inhibit VEGF driven capillary endothelial cell proliferation. Thus, attenuation of the effects of VEGF introduces a rationale for treatment of macular edema from venous occlusive disease.

Corticosteroids, a class of substances with anti-inflammatory properties, have been demonstrated to inhibit the expression of the VEGF gene. Nauck M. et al., Induction of vascular endothelial growth factor by platelet-activating factor and platelet-derived growth factor is downregulated by corticosteroids, Am J Resp Cell Mol Biol 1997; 16:398-406. Additionally, corticosteroids can downregulate the induction of VEGF by the pro-inflammatory mediators PDGF and platelet-activating factor (PAF) in a time and dose-dependent manner. Nauck M. et al., Corticosteroids inhibit the expression of the vascular endothelial growth factor gene in human vascular smooth muscle cells, Euro J Pharmacol 1998; 341:309-315. Thus, corticosteroids can to downregulate VEGF production and reduce breakdown of the blood-retinal barrier. Certain steroids can also have antiangiogenic properties possibly due to attenuation of the effects of VEGF. It should be noted that although certain corticosteroids can apparently down regulate VEGF production there are a number of other physiological mechanisms by which corticosteroids can effect the pathogenesis of an ocular condition, such as macular edema.

Triamcinolone

Triamcinolone is a corticosteroid and it has been reported that a saline suspension of triamcinoline (1 mg triamcinolone acetonide in 0.1 ml saline) is non-toxic upon intravitreal injection. McCuen B. et al., The lack of toxicity of intravitreally administered triamcinolone acetonide, Am J Ophthalmol 1981; 91:785-788. Intravitreal triamcinolone has been used to treat proliferative vitreoretinopathy (Jonas J. et al., Intravitreal injection of crystalline cortisone as adjunctive treatment of proliferative vitreoretinopathy, Br J Ophthalmol 2000; 84:1064-1067), as well as choroidal neovascularization (Challa J. et al., Exudative macular degeneration and intravitreal triamcinolone: 18 month follow up, Aust NZ J Ophthalmol 1998; 26:277-281; Penfold P. et al., Exudative macular degeneration and intravitreal triamcinolone: A pilot study, Aust NZ J Ophthalmol 1995; 23:293-298, and; Danis R. et al., Intravitreal triamcinolone acetonide in exudative age-related macular degeneration, Retina 2000; 20:244-250).

Additionally, European patent application 244 178 A2 (Keller) discloses intravitreal injection of an aqueous solution of dexamethasone and a hyaluronic acid, and a topical triamcinolone suspension for ear treatment is discussed in Chang H. et al., Development of a topical suspension containing three active ingredient, Drug Dev and Ind Pharm, 28(1), 29-39 (2002). Einmahl S. et al, Evaluation of a novel biomaterial in the suprachoroidal space of the rabbit eye, Invest Ophthal & Vis Sci 43(5); 1533-1539 (2002) discusses injection of a poly(ortho ester) into the suprachoroidal space, and Einmahl S. et al, Therapeutic applications of viscous and injectable poly(ortho esters), Adv Drug Del Rev 53 (2001) 45-73, discloses that a poly ortho ester polymer containing fluorouracil markedly degrades five days after intravitreal administration. See also U.S. Pat. No. 5,770,589 (Billson) which discusses intravitreal injection of a corticosteroid, such as triamcinolone acetonide. U.S. Pat. No. 5,209,926 (Babcock) discusses ophthalmic use of various amino substituted steroids.

Known formulations of triamcinolone clear (diffuses out of and/or is removed by one or more active transport mechanisms) from the vitreous within at most about 90 days, although it has been speculated that with a known formulation (Kenalog) the triamcinolone may be detectable in the vitreous for no more than four months after intravitreal injection. Thus, McCuen B. et al. (1981) supra at page 786 noted that after three months no triamcinolone was visible in any treated eyes. Others have reported that the triamcinolone present in a saline or other aqueous suspension or solution is upon intravitreal administration cleared from the vitreous in about 21-41 days; using opthalmoscopic and spectrophotometric detection means to determine disappearance of the injected triamcinolone, in non-vitrectomized rabbit eyes the average clearance rate of intravitreally triamcinolone (0.5 mg) was 41 days, while in eyes having undergone vitrectomy or combination vitrectomy and lensectomy the average clearance rate was 17 days and 7 days, respectively. Schindler R. et al., The clearance of intravitreal triamcinolone acetonide, Am J Ophthalmol 1982; 93:415-417. Using high-performance liquid chromatography (HPLC) complete clearance of intravitreally injected triamcinolone (0.4 mg) in 24 rabbit eyes was observed by 21 days. Scholes G. et al., Clearance of triamcinolone from vitreous, Arch Ophthalmol 1985; 103:1567-1569.). Such rapid clearance from the vitreous can necessitate frequent re-administration (re-dosing) in order to effectively treat an ocular condition.

A triamcinolone pharmaceutical composition available under the trade name Kenalog® (Bristol-Myers-Squibb, Princeton N.J.) has been widely used off-label to treat various ocular conditions, including by intravitreal administration. Significantly, Kenalog® is approved by the U.S. Food and Drug Administration only for intramuscular or intrabursal use, but not for the treatment of any ocular conditions. Each milliliter (ml) of Kenalog® 40 composition comprises 40 milligrams (mg) of triamcinolone acetonide, sodium chloride as a tonicity agent, 10 mg (0.99%) benzyl alcohol as a preservative, 7.5 mg (0.75%) of carboxymethylcellulose sodium and 0.4 mg (0.04%) of polysorbate 80 as resuspension aids.

It has been reported that Kenalog has a 15 day half life in the vitreous with an effect on central macular thickness being observed for up to 140 days after intravitreal injection of the Kenalog. Aubren, F. et al., Pharmacokinetic-Pharmacodynamic modeling of the effect of Triamcinolone Acetonide on Central Macular Thickness in Patients with Diabetic Macular Edema, Inv Ophth & Vis Sci, 45(10); 3435-3441: October 2004. It has also been reported that triamcinolone can be detected in the vitreous up to 93 days after a single intravitreal injection of Kenalog (Beer P. et al., Intraocular concentration and pharmacokinetics of triamcinolone acetonide after a single intravitreal injection, Opthal 110(4); 681-686: April 2003), with the triamcinolone estimated to be potentially detectable in the vitreous for about 4 months. Inoue M. et al., Vitreous concentrations of triamcinolone acetonide in human eyes after intravitreal or subtenon injection, Am J Opth 138(6); 1046-1048: 2004.

Noninfectious endophthalmitis have been reported upon intravitreal Kenalog® injection, possibly related to the preservative, excipients and/or resuspension aids present in Kenalog® (Roth D. et al., Noninfectious endophthalmitis associated with intravitreal triamcinolone injection, Arch Opthalmol 2003; 121: 1279-1282; Sutter F. et al., Pseudo-endophthalmitis after intravitreal injection of triamcinolone, Br J Ophthalmol 2003; 87:972-974).

Additionally, the presence of benzyl alcohol preservative and polysorbate 80 surfactant in Kenalog® can potentially damage or be toxic to sensitive ocular tissues, such as retinal cells, and for this reason clinicians routinely wash the triamcinolone acetonide precipitate (which forms when Kenalog® is left standing) several times with saline to reduce the concentration of these undesirable non-active materials from the formulation. Additionally, methods have been developed to filter out of Kenalog® and from identical formulations such as Kenacort-A the preservative, surfactant, and/or resuspension (suspending agents) aids present in these formulations. Nishimura A. et al., Isolating Triamcinolone acetonide particles for intravitreal use with a porous membrane filter, Retina, vol 23(6); 777-779 (2003). Such washing and/or filtering steps are inconvenient, time consuming, and increase the possibility of microbial or endotoxin contamination that could lead to intraocular infection and inflammation.

Significantly, the triamcinolone acetonide in Kenalog® 40 tends to rapidly separate and precipitate from the remainder of the composition. For example, if Kenalog® is left standing for as short a time as about five to ten minutes a substantial separation of a triamcinolone acetonide precipitate from the remainder of the composition occurs. Unfortunately, such rapid settling of the triamcinolone also occurs with other known saline based suspensions of triamcinolone (with or with preservatives and stabilizers). Thus, if the composition is to be injected into the eye it must first be vigorously shaken and used promptly after being so shaken in order to provide a substantially uniform suspension. A substantially uniform suspension (which is not provided by Kenalog or other saline based suspensions of triamcinolone) is required in order to provide a consistent and accurate dose upon administration of the suspension to the eye. In addition, resuspension processing requires the use of the resuspension aids noted above, at least one of which is less than totally desirable for sensitive ocular tissues. At least because of the potential risk of noninfectious endophthalmitis from use of the Kenalog® vehicle, development of a preservative-free triamcinolone formulation for intraocular use to treat an ocular condition (such as a posterior ocular condition) is desirable.

Elevated intraocular pressure, that is elevated anterior chamber intraocular pressure, depends on the comparative rates of aqueous production and aqueous drainage, primarily through the trabecular meshwork. Increased intraocular pressure occurs from a variety of mechanisms such as primary or secondary angle-closure glaucoma, primary or secondary open-angle glaucoma, or combined-mechanism glaucoma. If inadequately treated, increased intraocular pressure may result in glaucomatous optic nerve changes and loss of visual field.

Known formulations of corticosteroids administered by a topical, systemic or peribulbar route can cause an increase in anterior chamber intraocular pressure. For example, following 4 to 6 weeks of topical corticosteroid administration, 5% of subjects can show an elevation in intraocular pressure of >16 mmHg and 30% of subjects may show an elevation of 6 to 15 mmHg (Armaly M., Statistical attributes of the steroid hypertensive response in the clinically normal eye, Invest Ophthalmol Vis Sci 1965; 4:187-197; Becker B,. Intraocular pressure response to topical corticosteroid, Invest Ophthalmol Vis Sci 1965; 4:198-205). Additionally, intravitreal administration of known formulations of a corticosteroid, such as triamcinolone can also result in increased intraocular pressure (Martidis A. et al., Intravitreal triamcinolone for refractory diabetic macular edema, Ophthalmology 2002; 109:920-927; Jonas J. et al., Intravitreal injection of triamcinolone for diffuse diabetic macular edema, Arch Ophthalmol 2003; 121:57-61), possibly due to the burst or high release rates of triamcinolone from the known formulations.

As well as causing an increase in intraocular pressure, corticosteroids can also cause an increase in cataract formation. Corticosteroid-induced cataracts typically show an axial, posterior subcapsular opacity, which gradually increases in size. Nuclear sclerosis is not a typical lens change from corticosteroids. Topical, systemic and peribulbar corticosteroid administration have all been associated with an increased risk of cataract formation (Butcher J. et al., Bilateral cataracts and glaucoma induced by long term use of steroid eye drops. BMJ 1994; 309-343).

The intravitreal administration of known triamcinolone formulations can therefore also be expected to be associated with an increased risk of both elevated intraocular pressure and cataract formation.

A further adverse effect from ocular corticosteroid administration can be inflammation. Endophthalmitis is a type of intraocular inflammation that can be due to infection with pathogens such as bacteria of fungi or can be noninfectious. Clinical features include lid edema, conjunctival injection, corneal edema, anterior chamber and vitreous inflammation and hypotonia. Infectious endophthalmitis can occur following an intraocular procedure (i.e. cataract surgery, vitrectomy surgery, intravitreal injection), as a result of systemic infection, as a result of trauma, or occur as a late feature of conjunctival filtering blebs.

The most common dose of triamcinolone used to treat eyes with macular edema associated with diabetes, CRVO or BRVO is 4 mg (Martidis A. et al., Intravitreal triamcinolone for refractory diabetic macular edema, Ophthalmology 2002; 109:920-927). The use of 25 mg of triamcinolone has less commonly been used to treat eyes with macular edema (Jonas J. et al., Intraocular injection of crystalline cortisone as adjunctive treatment of diabetic macular edema, Am J Ophthalmol 2001; 132:425-427).

Thus, there are significant drawbacks and deficiencies with the known triamcinolone formulations used by intravitreal administration to treat an ocular condition, including for example rapid clearance from the vitreous, elevated intraocular pressure, cataract formation, retinal toxicity, and intraocular inflammation, such as endophthalmitis.

Hence, a sterile, preservative-free, sustained release triamcinolone preparation is desirable. Additionally, because corticosteroids have known ocular toxicities (as manifested in the occurrence or development of for example elevated IOP, glaucoma and cataract) it is desirable to have a triamcinolone formulation for intraocular (i.e. intravitreal) use which does not result in an increased incidence of elevated IOP, glaucoma, cataract formation and/or intraocular inflammation, or which has, subsequent to intraocular administration of a triamcinolone formulation, a reduced incidence of elevated IOP, glaucoma, cataract formation and/or intraocular inflammation as compared to currently used or known intraocular (i.e. intravitreal) use triamcinolone.

### DRAWINGS

FIG. 1 is a bar graph of observed angiographic leakage (as assessed on a 1-5 grading scale) on the Y-axis versus time of the observation on the X-axis for three groups of rabbit eyes: rabbit control (untreated) eyes, rabbit eyes intravitreally injected with the 1 mg triamcinolone acetonide gel suspension (TAA_{gs}) formulation of Example 8, and rabbit eyes intravitreally injected with the 4 mg TAA_{gs} of Example 9. The grading (scale 1-5) of late-phase angiograms from rabbit eyes was measured over a thirty week period after intravitreal injection of either the 1 mg TAA_{gs} or 4 mg TAA_{gs}. All eyes received intravitreal injection of 500 ng VEGF at each of the time points shown on the X-axis followed by angiography 48 hrs later.

FIG. 2 is a bar graph of observed vitreoretinal fluorescence (as area under the curve) on the Y-axis versus time of the observation on the X-axis for the same three groups of rabbit eyes: rabbit control (untreated) eyes, rabbit eyes intravitreally injected with the 1 mg TAA_{gs}, and rabbit eyes intravitreally injected with the 4 mg TAA_{gs} (as in FIG. 1). Scanning vitreal fluorophotometry measurements of VEGF-induced BRB breakdown in rabbit eyes was measured over the same thirty week period after intravitreal injection of the 1 mg or 4 mg TAA_{gs}. As in FIG. 1, all eyes had received intravitreal injection of 500 ng VEGF at the time points shown on the X-axis followed by fluorophotometry 48 hrs later. The area under the fluorescence curve (AUC) was calculated for each eye.

FIG. 3 is a bar graph of observed retinal blood vessel caliber and tortuosity (grade) on the Y-axis versus time of the observation on the X-axis for the same rabbit control (untreated) eyes, rabbit eyes intravitreally injected with 1 mg TAA_{gs} or with 4 mg TAA_{gs} (as in FIG. 1). Subjective grading (on a 1-5 scale) of VEGF-induced changes in vessel caliber and tortuosity from fundus images of rabbit eyes was measured over the same thirty week period after intravitreal injection of the 1 or 4 mg TAA_{gs}. As in FIG. 1, all eyes received intravitreal injection of 500 ng VEGF at the indicated time points followed by fundus image capture 48 hrs later.

FIG. 4 is a bar graph of observed anterior chamber fluorescence (as area under the curve) on the Y-axis versus time of the observation on the X-axis for the same rabbit control (untreated) eyes, rabbit eyes intravitreally injected with 1 mg TAA_{gs} or with 4 mg TAA_{gs}, (as in FIG. 1). Scanning ocular fluorophotometry of VEGF-induced blood-aqueous barrier breakdown in rabbit eyes was measured over a thirty week period after intravitreal injection of either 1 or 4 mg TAA_{gs}. As in FIG. 1, all eyes received intravitreal injection of 500 ng VEGF at the indicated time points followed by anterior chamber fluorophotometry 48 hrs later. The area under the fluorescence curve (AUC) was calculated for each eye.

FIG. 5 is a negative image of a photograph of the eye of a rabbit thirty weeks after intravitreal injection of 50 μL of the Example 9 4 mg TAA_{gs} formulation. The photograph was taken with an 11.0 megapixel, digital Zeiss FF450 fundus camera coupled to the Zeiss 481 Visupac image capture and analysis system.

FIG. 6 is a flow chart which summarizes a preferred manufacturing process for making the triamcinolone formulations of Examples 1 to 9.

### SUMMARY

The present invention provides sterile, preservative-free, sustained release triamcinolone formulations for treating ocular conditions with the desirable characteristics of low ocular toxicities, as manifested in the low or nominal occurrence or development of an elevated IOP, glaucoma, cataract and/or intraocular inflammation.

Definitions

As used herein, the words or terms set forth below have the following definitions.

“About” means that the item, parameter or term so qualified encompasses a range of plus or minus ten percent above and below the value of the stated item, parameter or term.

“Administration”, or “to administer” means the step of giving (i.e. administering) a pharmaceutical composition to a subject. The pharmaceutical compositions disclosed herein can be “locally administered”, that is administered at or in the vicinity of the site at which a therapeutic result or outcome is desired. For example to treat an ocular condition (such as for example a macular edema, uveitis or macular degeneration) intravitreal injection or implantation of a sustained release device such as active agent containing polymeric implant can be carried out. “Sustained release” means release of an active agent (such as a triamcinolone) over a period of about seven days or more, while “extended release” means release of an active agent over a period of time of less than about seven days.

“Entirely free (i.e. “consisting of” terminology) means that within the detection range of the instrument or process being used, the substance cannot be detected or its presence cannot be confirmed.

“Essentially free” (or “consisting essentially of”) means that only trace amounts of the substance can be detected.

“Pharmaceutical composition” means a formulation in which an active ingredient (the active agent) can be a steroid, such as a corticosteroid, such as a triamcinolone. The word “formulation” means that there is at least one additional ingredient in the pharmaceutical composition besides the steroid active ingredient. A pharmaceutical composition is therefore a formulation which is suitable for diagnostic or therapeutic administration (i.e. by intraocular injection or by insertion of a depot or implant) to a subject, such as a human patient.

“Substantially free” means present at a level of less than one percent by weight of the pharmaceutical composition.

The present compositions are highly suitable for intravitreal administration into the posterior segments of eyes without requiring any washing step, while providing for reduced ocular, for example, retinal, damage when used in an eye. The present compositions are advantageously substantially free of added preservative components, for example, contain no benzyl alcohol preservative. In addition, the present compositions advantageously require no resuspension aid or aids. Overall, the present compositions are easily and effectively injectable into the posterior segment of an eye of a human or animal and can be maintained as a substantially uniform suspension for long periods of time, for example, at least about one week or more, without resuspension processing, for example, without requiring shaking or other agitating of the composition to obtain substantial suspension uniformity. In short, the present compositions and methods provide substantial enhancements and advantages, for example, relative to the prior art Kenalog® 40 composition and methods of using such prior art composition, in the posterior segments of human or animal eyes.

In one broad aspect of the present invention, compositions useful for injection into a posterior segment of an eye of a human or animal are provided. Such compositions comprise a corticosteroid component, a viscosity inducing component, and an aqueous carrier component. The corticosteroid component is present in a therapeutically effective amount. The corticosteroid component is present in the compositions in a plurality of particles.

The present compositions may include a corticosteroid component in an amount of up to about 25% (w/v) or more of the composition. In one very useful embodiment, the corticosteroid component is present in an amount of at least about 80 mg/ml of composition. Preferably, the corticosteroid component is present in an amount in a range of about 1% to about 10% or about 20% (w/v) of the composition.

In one very useful embodiment, the corticosteroid component comprises triamcinolone acetonide. The viscosity inducing component is present in an amount effective in increasing the viscosity of the composition. Any suitable, preferably ophthalmically acceptable, viscosity inducing component may be employed in accordance with the present invention. Many such viscosity inducing components have been proposed and/or used in ophthalmic compositions used on or in the eye. Advantageously, the viscosity inducing component is present in an amount in a range of about 0.5% to about 20% (w/v) of the composition. In one particularly useful embodiment, the viscosity inducing component is a hyaluronic acid polymer component, such as sodium hyaluronate.

In one embodiment, the present compositions have a viscosity of at least about 10 cps or at least about 100 cps, preferably at least about 1,000 cps, more preferably at least about 10,000 cps and still more preferably at least about 70,000 cps, for example, up to about 250,000 cps, or about 300,000 cps, at a shear rate of 0.1/second. The present compositions are structured or have make-ups so as to be effectively, for example, manually, injected into a posterior segment of an eye of a human or animal, preferably through a 27 gauge needle, more preferably through a 29 or 30 gauge needle.

Without wishing to limit the invention to any particular theory of operation, it is believed that the use of relatively high viscosity compositions, as described herein, provides for effective, and preferably substantially uniform, suspension of the steroid component particles while, at the same time, being injectable into the posterior segment of an eye through conventionally, or even smaller than conventionally, used needles.

In one embodiment of the invention, the corticosteroid component is present in a plurality of particles which are substantially uniformly suspended in the composition and remain substantially uniformly suspended in the composition for at least about 1 week, preferably at least about 2 weeks or at least about 1 month, and still more preferably at least about 6 months or at least about 1 year or at least about 2 years, without requiring resuspension processing, that is, without requiring being shaken or otherwise agitated to maintain the corticosteroid component particles substantially uniformly suspended in the composition.

Compositions having such substantially uniform suspension of corticosteroid component particles, so as to be able to provide a consistent and accurate dose upon administration to an eye, provide substantial advantages relative to the prior art. In particular, the present compositions may be manufactured, shipped and stored for substantial periods of time without the corticosteroid component particles precipitating from the remainder of the composition. Having the corticosteroid component particles maintained substantially uniformly suspended in the composition allows the composition to provide long term dosing consistency and accuracy per unit dose amount administered, without any need to resuspend the corticosteroid particles.

The aqueous carrier component is advantageously ophthalmically acceptable and may include one or more conventional expedients useful in ophthalmic compositions. For example, the carrier component may include an effective amount of at least one of a preservative component, a tonicity component and a buffer component. In one advantageous embodiment, the present compositions include no added preservative component. This feature reduces or minimizes or even substantially eliminates adverse reactions in the eye which may be caused by or linked to the presence of a preservative component. Although a resuspension component may be employed in accordance with the present invention, in many instances, because of the ability of the present composition to remain a substantially uniform suspension for a long period of time without requiring resuspension processing, the compositions advantageously contain no added resuspension components.

Methods of treating posterior segments of the eyes of humans or animals are also disclosed and are included within the scope of the present invention. In general, such methods comprise administering, e.g. injecting a corticosteroid component-containing composition, for example, a composition in accordance with the present intention, to a posterior segment of an eye of a human or animal. Such administering is effective in providing a desired therapeutic effect. The administering step advantageously comprises at least one of intravitreal injecting, subconjunctival injecting, sub-tenon injecting, retrobulbar injecting, suprachoroidal injecting and the like.

Our invention encompasses a pharmaceutical composition for treating a posterior ocular condition. The composition can comprise a triamcinolone present in a therapeutically effective amount as a plurality of particles; a viscosity inducing component in an amount effective to increase the viscosity of the composition, and; an aqueous carrier component. The composition can have a viscosity of at least about 10 cps at a shear rate of about 0.1/second and is injectable into the vitreous of a human eye, for example through a 27 gauge needle. By reducing the viscosity of our formulation it can be injected into the vitreous through a 28, 29 or 30 gauge needle.

Preferably, the triamcinolone particles of the pharmaceutical composition are substantially uniformly suspended in the composition and the viscosity inducing component is a polymeric hyaluronate.

A detailed embodiment within the scope of our invention is a pharmaceutical composition for treating a posterior ocular condition, comprising triamcinolone particles; polymeric hyaluronate, in which the triamcinolone particles are suspended; sodium chloride; sodium phosphate, and water. The pharmaceutical composition can have a viscosity at a shear rate of about 0.1/second of between about 80,000 cps to about 300,000, preferably from about 100,000 cps to about 300,000 cps, and most preferably from about 1280,000 cps to about 225,000 cps. Note that the pharmaceutical composition can have a viscosity at a shear rate of about 0.1/second of between about 80,000 cps and about 300,000 cps, and that when the pharmaceutical composition has a viscosity at a shear rate of about 0.1/second of between about 100,000 cps and about 150,000 cps it can be injected into the vitreous through a 27, 28, 29 or 30 gauge needle. We have found that even with a 300,000 cps our formulations can be injected through a 30 gauge needle due to shear thinning once the formulation is in movement in the syringe. The sodium phosphate present in the pharmaceutical composition can comprise both monobasic sodium phosphate and dibasic sodium phosphate. Additionally, the pharmaceutical composition can comprise between about 2% w/v triamcinolone and about 8% w/v triamcinolone, between about 2% w/v hyaluronate and about 3% w/v hyaluronate, about 0.6% w/v sodium chloride and between about 0.03% w/v sodium phosphate and about 0.04% w/v sodium phosphate. Alternately, the pharmaceutical composition of claim 5 can comprise between about 0.5% w/v hyaluronate and about 6% w/v hyaluronate. If desired the hyaluronate can be heated (see Example 15) to decrease its molecular weight (and therefore its viscosity) in the formulation.

The pharmaceutical composition can also comprises between about 0.6% w/v sodium chloride to about 0.9% w/v sodium chloride. Generally, more sodium chloride is used in the formulation as less phosphate is used in the formulation, for example 0.9% sodium chloride can be used if no phosphate is present in the formulation, as in this manner the tonicity of the formulation can be adjusted to obtain the desired isotonicity with physiological fluid. The pharmaceutical composition can comprise between about 0.0% w/v sodium phosphate and 0.1% w/v sodium phosphate. As noted, more phosphate can be used in the formulation if less sodium chloride is present in the formulation so as to obtain a desired pH 7.4 buffering effect.

A more detailed embodiment within the scope of our invention is a pharmaceutical composition for treating a posterior ocular condition, the pharmaceutical composition consisting essentially of triamcinolone particles, polymeric hyaluronate, in which polymeric hyaluronate the triamcinolone particles are suspended, sodium chloride, sodium phosphate, and water. The pharmaceutical composition can have a viscosity at a shear rate 0.1/second of between about 128,000 cps and about 225,000 cps and the sodium phosphate present in the pharmaceutical composition can be present as both monobasic sodium phosphate and dibasic sodium phosphate.

A further embodiment of our invention is a triamcinolone suspension for treating a posterior ocular condition, consisting of triamcinolone particles, polymeric hyaluronate, in which the triamcinolone particles are suspended, sodium chloride, dibasic sodium phosphate heptahydrate, monobasic sodium phosphate monohydrate, and water, wherein the composition has a viscosity at a shear rate 0.1/second of between about 128,000 cps and about 225,000 cps.

Our invention also includes a method for treating a posterior ocular condition by administering (as by injecting) the pharmaceutical composition of claim 1 to the vitreous of a human or animal, thereby treating the posterior ocular condition. Thus we have invented a method for treating macula edema by administering to the vitreous of a human eye a pharmaceutical composition comprising a triamcinolone, and a hyaluronate, wherein the pharmaceutical composition having a viscosity at a shear rate 0.1/second of between about 128,000 cps and about 225,000 cps.

A pharmaceutical composition within the scope of our invention for treating a posterior ocular condition can comprise a triamcinolone present in a therapeutically effective amount as a plurality of particles, a viscosity inducing component in an amount effective to increase the viscosity of the composition, and an aqueous carrier component, wherein the composition has a viscosity of at least about 10 cps at a shear rate of 0.1/second and is injectable into the vitreous of a human eye and wherein the pharmaceutical composition releases the triamcinolone with substantially first order release kinetics over a period of at least about 45 days after the intravitreal injection. This pharmaceutical composition can exhibit reduced generation of intraocular inflammation, no plume effect (that is no wide dispersion of the triamcinolone into the vitreous as soon as the triamcinolone is intravitreally injected), and cohesiveness (as shown by the retention of the form of the triamcinolone gel for 30 weeks or longer after intravitreal injection of the triamcinolone gel formulation) upon intravitreal injection of the pharmaceutical composition.

Our invention encompasses a method for treating a posterior ocular condition, the method comprising the step of intravitreal administration of a sustained release pharmaceutical composition implant comprising a triamcinolone present in a therapeutically effective amount as a plurality of particles, a viscosity inducing component in an amount effective to increase the viscosity of the composition, and an aqueous carrier component, wherein the composition has a viscosity of at least about 10 cps at a shear rate of 0.1/second and is injectable into the vitreous of a human eye, and wherein the posterior ocular condition is treated for up to about 30 weeks by the triamcinolone released from the implant. In this method the pharmaceutical composition can comprise triamcinolone particles, polymeric hyaluronate, in which the triamcinolone particles are suspended, sodium chloride, sodium phosphate, and water. Additionally, the intravitreal administration can be injected through a 27 gauge needle into the vitreous of a human eye, and in an aggregate number of patients practise of the method results in less intraocular inflammation than does practise of the same method with a second pharmaceutical composition which is a saline solution or suspension of a triamcinolone.

Finally, our invention also includes a process for making a pharmaceutical composition by (a) mixing triamcinolone particles about 4 microns to about 8 microns in diameter with sodium chloride crystals, and about 35% to about 40% of the total volume of the water (water for injection) used to make the formulation; (b) heating the triamcinolone and sodium chloride mixture to a temperature between about 120° C. and about 140° C., thereby preparing a first part; (c) mixing a sodium phosphate and water, thereby preparing a second part; (d) dissolving sodium hyaluronate with a molecular weight between about 1.0 million Daltons and about 1.9 million Daltons in another about 35% to about 40% of the total water volume used to make the formulation, followed by sterile filtration after the dissolving; (e) lyophilization of the dissolved sodium hyaluronate; (f) reconstitution of the lyophilized, sterile sodium hyaluronate, thereby preparing a third part; and; (g) aseptically combining the first, second and third parts, thereby making a sterile, uniform triamcinolone pharmaceutical composition which is, an opaque white gel suspension suitable for intravitreal injection to treat an ocular condition. Water is added as needed (q.s.) to make the desired gel suspension which is about 80% to about 90% by weight water.

### DESCRIPTION

The present invention is based upon our discovery of triamcinolone formulations specifically designed for intravitreal injection to treat various ocular conditions, such a macula edema. Our triamcinolone formulations have numerous superior characteristics and advantages, including the following: (1) the triamcinolone present in our formulations does not rapidly settle out from or precipitate from the formulations. Importantly our formulations have a shelf life of at least two years, meaning that our formulations can be left standing for up to about two years before administration to an eye, and after two years the formulation can still provide a consistent and accurate dose of triamcinolone upon injection to the formulation to an eye; (2) our formulations are free of preservatives and resuspension aids, such as benzyl alcohol and/or a polysorbate; (3) concomitantly, our formulations have a much reduced retinal and photoreceptor toxicity; (4) as well as being sterile and preservative-free our triamcinolone formulations can provide sustained release of therapeutic amounts of the triamcinolone over multi-month periods upon intravitreal injection of such formulations. Thus, our viscous suspension triamcinolone formulations can be characterized as sustained release implants; (5) intravitreal administration of our triamcinolone formulations is not associated with an increased incidence of adverse events such as elevated intra ocular pressure, glaucoma, cataract and/an intraocular inflammation; (6) intravitreal administration of our triamcinolone formulations is not associated with an increased incidence of adverse events such elevated intra ocular pressure, glaucoma, cataract and/an intraocular inflammation as compared to currently used or known intraocular (i.e. intravitreal) use triamcinolone formulations; (7) our formulations permit triamcinolone particles (crystals) to be released (as they solubilize in the viscous fluid of the posterior chamber) from a discrete unitary location, thereby avoiding the plume effect (rapid dispersion) characteristic of aqueous triamcinolone formulations upon intravitreal administration; (8) avoidance of plume formation or rapid dispersion upon intravitreal administration beneficially reduces visual field obscuration; (9) the sustained release characteristic of our formulations reduces the need for intravitreal administration of large drug quantities to achieve a desired therapeutic effect; (10) upon intravitreal administration, the triamcinolone present in our formulations can preferentially be eliminated in posterior direction (that is through the retina and optic nerve) as opposed to elimination through an anterior route (see eg Table 5). This can result in superior treatment of a retinal disease with for example reduced ocular hypertension.

Advantage (1) above can be provided by formulating the triamcinolone as a viscous, gel suspension, as opposed to formulating it as an aqueous or saline based formulation. Additionally, advantages (4) and (8) above can be provided by particular characteristics of our formulations, such as suspension of the triamcinolone in one or more particular high molecular weight hydrogel polymers which permit sustained release of the triamcinolone from a biocompatible, biodegradable polymeric matrix, and other implant-like characteristics to the formulations, including substantially zero-order in vivo (i.e. intravitreal) release kinetics (see eg Table 4).

The pluming effect occurs when a saline suspension of a triamcinolone (such as Kenalog) is injected into the vitreous. Pluming prevents visualization of the back of the eye (i.e. the retina is obscured) and also reduces the patient's visual field (reduced vision).

Generally, the present invention provides compositions useful for placement, preferably by injection, into a posterior segment of an eye of a human or animal. Such compositions in the posterior, e.g., vitreous, of the eye are therapeutically effective against one or more conditions and/or diseases of the posterior of the eye, and/or one or more symptoms of such conditions and/or diseases of the posterior of the eye.

It is important to note that while preferably the compositions disclosed herein are preferably administered by intravitreal injection to treat a posterior ocular condition, our compositions (i.e. those of Examples 8 and 9) can also be administered (as by injection) by other routes, such as for example subconjuctival, sub-tenon, periocular, retrobulbar, suprachoroidal, and/or intrascleral to effectively treat an ocular condition. Additionally, a sutured on refillable dome can be placed over the administration site to prevent or to reduce wash out, leaching and/or diffusion of the active agent in a non-preferred direction.

Compositions within the scope of our invention can comprise a corticosteroid component; a viscosity inducing component; and an aqueous carrier component. The compositions are advantageously ophthalmically acceptable. One of the important advantages of the present compositions is that they are more compatible with or friendly to the tissues in the posterior segment of the eye, for example, the retina of the eye, relative to compositions previously proposed for intravitreal injection into a posterior segment of an eye, for example, a composition sold under the trademark Kenalog®-40. In particular, the present compositions advantageously are substantially free of added preservative components or include effective preservative components which are more compatible with or friendly to the posterior segment, e.g., retina, of the eye relative to benzyl alcohol, which is included in the Kenalog®-40 composition as a preservative.

In addition, the present compositions preferably include no added resuspension component, such as polysorbate-80, which is included in the Kenalog®-40 composition. Many of the other features of the present compositions, as described elsewhere herein, also render the present compositions more compatible with or friendly to the posterior segments of the eyes into which the compositions are placed relative to prior art compositions, such as Kenalog®-40.

As noted above, the present compositions include a corticosteroid component. Such corticosteroid component is present in the compositions in a therapeutically effective amount, that is in an amount effective in providing a desired therapeutic effect in the eye into which the composition is placed. The corticosteroid component is present in the composition in a plurality of particles. Any suitable corticosteroid component may be employed in according to the present invention. Such corticosteroid component advantageously has a limited solubility in water, for example, at 25° C. For example, the corticosteroid component preferably has a solubility in water at 25° C. of less than 10 mg/ml. Of course, the corticosteroid component should be ophthalmically acceptable, that is, should have substantially no significant or undue detrimental effect of the eye structures or tissues. One particularly useful characteristic of the presently useful corticosteroid components is the ability of such component to reduce inflammation in the posterior segment of the eye into which the composition is placed caused by the result of one or more diseases and/or conditions in the posterior segment of the eye.

Examples of useful corticosteroid components include, without limitation, cortisone, prednesolone, triamcinolone, triamcinolone acetonide, fluorometholone, dexamethosone, medrysone, loteprednol, derivatives thereof and mixtures thereof. As used herein, the term “derivative” refers to any substance which is sufficiently structurally similar to the material of which it is identified as a derivative so as to have substantially similar functionality or activity, for example, therapeutic effectiveness, as the material when the substance is used in place of the material. In one very useful embodiment, the corticosteroid component comprises triamcinolone acetonide.

The corticosteroid component advantageously is present in an amount of at least about 10 mg per ml of the composition. One important advantage of the present invention is the effective ability of the present compositions to include relatively large amounts or concentrations of the corticosteroid component. Thus, the corticosteroid component may be present in the present compositions in an amount in the range of about 1 % or less to about 5% or about 10% or about 20% or about 30% or more (w/v) of the composition. Providing relatively high concentrations or amounts of corticosteroid component in the present compositions is beneficial in that reduced amounts (volumes for injection) of the composition may be required to be placed or injected into the posterior segment of the eye in order to provide the same amount or more corticosteroid component in the posterior segment of the eye relative to compositions, such as Kenalog®-40, which include less than 4% (w/v) of the corticosteroid component. Thus, in one very useful embodiment, the present compositions include more than about 4% (w/v), for example at least about 5% (w/v), to about 10% (w/v) or about 20% (w/v) or about 30% (w/v) of the corticosteroid component. For example, about 50 pL of our Example 8 or 9 formulation provide respectively 2 mg and 4 mg of triamcinolone. This is in contrast to other formulations (such as Kenalog 40) which require 100 μL to provide 4 mg of triamcinolone. Injection of 100 μL or more of a fluid into the vitreous can result in an excess of fluid in the vitreous with elevated intraocular pressure and leakage of the fluid from the vitreous then potentially occurring.

The viscosity inducing component is present in an effective amount in increasing, advantageously substantially increasing, the viscosity of the composition. Without wishing to limit the invention to any particular theory of operation, it is believed that increasing the viscosity of the compositions to values well in excess of the viscosity of water, for example, at least about 100 cps at a shear rate of 0.1/second, compositions which are highly effective for placement, e.g., injection, into the posterior segment of an eye of a human or animal are obtained. Along with the advantageous placement or injectability of the present compositions into the posterior segment, the relatively high viscosity of the present compositions are believed to enhance the ability of the present compositions to maintain the corticosteroid component particles in substantially uniform suspension in the compositions for prolonged periods of time, for example, for as long as 1 to 2 years, without requiring resuspension processing. The relatively high viscosity of the present compositions may also have an additional benefit of at least assisting the compositions to have the ability to have an increased amount or concentration of the corticosteroid component, as discussed elsewhere herein, for example, while maintaining such corticosteroid component in substantially uniform suspension for prolonged periods of time.

Advantageously, the present compositions have viscosities of at least about 10 cps or at least about 100 cps or at least about 1000 cps, more preferably at least about 10,000 cps and still more preferably at least about 70,000 cps or more, for example up to about 200,000 cps or about 250,000 cps, or about 300,000 cps or more, at a shear rate of 0.1/second. The present compositions not only have the relatively high viscosity as noted above but also have the ability or are structured or made up so as to be effectively placeable, e.g., injectable, into a posterior segment of an eye of a human or animal, preferably through a 27 gauge needle, or even through a 30 gauge needle.

The presently useful viscosity inducing components preferably are shear thinning components in that as the present composition containing such a shear thinning viscosity inducing component is passed or injected into the posterior segment of an eye, for example, through a narrow space, such as 27 gauge needle, under high shear conditions the viscosity of the composition is substantially reduced during such passage. After such passage, the composition regains substantially its pre-injection viscosity so as to maintain the corticosteroid component particles in suspension in the eye.

Any suitable viscosity inducing component, for example, ophthalmically acceptable viscosity inducing component, may be employed in accordance with the present invention. Many such viscosity inducing components have been proposed and/or used in ophthalmic compositions used on or in the eye. The viscosity inducing component is present in an amount effective in providing the desired viscosity to the composition. Advantageously, the viscosity inducing component is present in an amount in a range of about 0.5% or about 1.0% to about 5% or about 10% or about 20% (w/v) of the composition. The specific amount of the viscosity inducing component employed depends upon a number of factors including, for example and without limitation, the specific viscosity inducing component being employed, the molecular weight of the viscosity inducing component being employed, the viscosity desired for the present composition being produced and/or used and the like factors, such as shear thinning. The viscosity inducing component is chosen to provide at least one advantage, and preferably multiple advantages, to the present compositions, for example, in terms of each of injectability into the posterior segment of the eye, viscosity, sustainability of the corticosteroid component particles in suspension, for example, in substantially uniform suspension, for a prolonged period of time without resuspension processing, compatibility with the tissues in the posterior segment of the eye into which the composition is to be placed and the like advantages. More preferably, the selected viscosity inducing component is effective to provide two or more of the above-noted benefits, and still more preferably to provide all of the above-noted benefits.

The viscosity inducing component preferably comprises a polymeric component and/or at least one viscoelastic agent, such as those materials which are useful in ophthalmic surgical procedures.

Examples of useful viscosity inducing components include, but are not limited to, hyaluronic acid (such as a polymeric hyaluronic acid), carbomers, polyacrylic acid, cellulosic derivatives, polycarbophil, polyvinylpyrrolidone, gelatin, dextrin, polysaccharides, polyacrylamide, polyvinyl alcohol, polyvinyl acetate, derivatives thereof and mixtures and copolymers thereof.

The molecular weight of the presently useful viscosity inducing components may be in a range of about 10,000 Daltons or less to about 2 million Daltons or more. In one particularly useful embodiment, the molecular weight of the viscosity inducing component is in a range of about 100,000 Daltons or about 200,000 Daltons to about 1 million Daltons or about 1.5 million Daltons. Again, the molecular weight of the viscosity inducing component useful in accordance with the present invention, may vary over a substantial range based on the type of viscosity inducing component employed, and the desired final viscosity of the present composition in question, as well as, possibly one or more other factors.

In one very useful embodiment, a viscosity inducing component is a polymeric hyaluronate component, for example, a metal hyaluronate component, preferably selected from alkali metal hyaluronates, alkaline earth metal hyaluronates and mixtures thereof, and still more preferably selected from sodium hyaluronates, and mixtures thereof. The molecular weight of such hyaluronate component (i.e. a polymeric hyaluronic acid) preferably is in a range of about 50,000 Daltons or about 100,000 Daltons to about 1.3 million Daltons or about 2 million Daltons. In one embodiment, the present compositions include a polymeric hyaluronate component in an amount in a range about 0.05% to about 0.5% (w/v). In a further useful embodiment, the hyaluronate component is present in an amount in a range of about 1% to about 4% (w/v) of the composition. In this latter case, the very high polymer viscosity forms a gel that slows particle sedimentation rate to the extent that often no resuspension processing is necessary over the estimated shelf life, for example, at least about 2 years, of the composition. Such a composition may be marketed in pre-filled syringes since the gel cannot be easily removed by a needle and syringe from a bulk container. Pre-filled syringes have the advantages of convenience for the injector and the safety which results from less handling.

The aqueous carrier component is advantageously ophthalmically acceptable and may include one or more conventional excipients useful in ophthalmic compositions. The present compositions preferably include a major amount of liquid water. The present compositions may be, and are preferably, sterile, for example, prior to being used in the eye.

The present compositions preferably include at least one buffer component in an amount effective to control the pH of the composition and/or at least one tonicity component in an amount effective to control the tonicity or osmolality of the compositions. More preferably, the present compositions include both a buffer component and a tonicity component.

The buffer component and tonicity component may be chosen from those which are conventional and well known in the ophthalmic art. Examples of such buffer components include, but are not limited to, acetate buffers, citrate buffers, phosphate buffers, borate buffers and the like and mixtures thereof. Phosphate buffers are particularly useful. Useful tonicity components include, but are not limited to, salts, particularly sodium chloride, potassium chloride, mannitol and other sugar alcohols, and other suitable ophthalmically acceptably tonicity component and mixtures thereof.

The amount of buffer component employed preferably is sufficient to maintain the pH of the composition in a range of about 6 to about 8, more preferably about 7 to about 7.5. The amount of tonicity component employed preferably is sufficient to provide an osmolality to the present compositions in a range of about 200 to about 400, more preferably about 250 to about 350, mOsmol/kg respectively. Advantageously, the present compositions are substantially isotonic.

The present compositions may include one or more other components in amounts effective to provide one or more useful properties and/or benefits to the present compositions. For example, although the present compositions may be substantially free of added preservative components, in other embodiments, the present compositions include effective amounts of preservative components, preferably such components which are more compatible with or friendly to the tissue in the posterior segment of the eye into which the composition is placed than benzyl alcohol. Examples of such preservative components include, without limitation, benzalkonium chloride, chlorhexidine, PHMB (polyhexamethylene biguanide), methyl and ethyl parabens, hexetidine, chlorite components, such as stabilized chlorine dioxide, metal chlorites and the like, other ophthalmically acceptable preservatives and the like and mixtures thereof. The concentration of the preservative component, if any, in the present compositions is a concentration effective to preserve the composition, and is often in a range of about 0.00001% to about 0.05% or about 0.1% (w/v) of the composition.

In addition, the present composition may include an effective amount of resuspension component effective to facilitate the suspension or resuspension of the corticosteroid component particles in the present compositions. As noted above, in certain embodiments, the present compositions are free of added resuspension components. In other embodiments of the present compositions effective amounts of resuspension components are employed, for example, to provide an added degree of insurance that the corticosteroid component particles remain in suspension, as desired and/or can be relatively easily resuspended in the present compositions, such resuspension be desired. Advantageously, the resuspension component employed in accordance with the present invention, if any, is chosen to be more compatible with or friendly to the tissue in the posterior segment of the eye into which the composition is placed than polysorbate 80.

Any suitable resuspension component may be employed in accordance with the present invention. Examples of such resuspension components include, without limitation, surfactants such as poloxanes, for example, sold under the trademark Pluronic®; tyloxapol; sarcosinates; polyethoxylated castor oils, other surfactants and the like and mixtures thereof.

One very useful class of resuspension components are those selected from vitamin derivatives. Although such materials have been previously suggested for use as surfactants in ophthalmic compositions, they have been found to be effective in the present compositions as resuspension components. Examples of useful vitamin derivatives include, without limitation, Vitamin E tocopheryl polyethylene glycol succinates, such as Vitamin E tocopheryl polyethylene glycol 1000 succinate (Vitamin E TPGS). Other useful vitamin derivatives include, again without limitation, Vitamin E tocopheryl polyethylene glycol succinamides, such as Vitamin E tocopheryl polyethylene glycol 1000 succinamide (Vitamin E TPGSA) wherein the ester bond between polyethylene glycol and succinic acid is replaced by an amide group.

The presently useful resuspension components are present, if at all, in the compositions in accordance with the present invention in an amount effective to facilitate suspending the particles in the present compositions, for example, during manufacture of the compositions or thereafter. The specific amount of resuspension component employed may vary over a wide range depending, for example, on the specific resuspension component being employed, the specific composition in which the resuspension component is being employed and the like factors. Suitable concentrations of the resuspension component, if any, in the present compositions are often in a range of about 0.01 % to about 5%, for example, about 0.02% or about 0.05% to about 1.0% (w/v) of the composition.

The availability of minimally soluble corticosteroid components, such as triamcinolone acetonide, to intraocular tissues may be limited by the dissolution rate for these substances. Slow dissolution is both good and bad for the patient. On the one hand, after a single intravitreal injection of the present composition, the mean elimination half-life for triamcinolone acetonide is advantageously quite long, for example, about 19 days in nonvitrectonized patients and measurable drug levels are detected for up to about 3 months. On the other hand, therapeutic drug levels in the vitreous compartment of the eye may not be achieved for about 1 to about 3 days, due to the slow dissolution rate of the corticosteroid component particles.

In one embodiment of the present invention, an effective amount of a solubilizing component is provided in the composition to solubilize a minor amount, that is less than 50%, for example in a range of 1% or about 5% to about 10% or about 20% of the corticosteroid component. For example, the inclusion of a cyclodextrin component, such as β-cyclodextrin, sulfo-butylether β-cyclodextrin (SBE) , other cyclodextrins and the like and mixtures thereof, at about 0.5 to about 5.0% (w/v) solubilizes about 1 to about 10% of the initial dose of triamcinolone acetonide. This presolubilized fraction provides a readily bioavailable loading dose, thereby avoiding any delay time in therapeutic effectiveness.

The use of such a solubilizing component is advantageous to provide any relatively quick release of the corticosteroid component into the eye for therapeutic effectiveness. Such solubilizing component, of course, should be ophthalmically acceptable or at least sufficiently compatible with the posterior segment of the eye into which the composition is placed to avoid undue damage to the tissue in such posterior segment.

The pharmacokinetics of the corticosteroid component, for example, triamcinolone acetonide, following intravitreal administration may involve both the rate of drug dissolution and the rate of drug efflux via the anterior route. For example, following a single intravitreal injection of a composition containing 4% (w/v) of triamcinolone acetonide, triamcinolone acetonide concentration peaks (monitored in aqueous humor) after several days at thousands of nanograms per mL. This peak (Cₘₐₓ) is followed by a rapid decrease lasting about 200 hours, and ends in a slow elimination phase with a half-life of about 19 days. Patients typically require repeat dosing, for example about every three months.

In one embodiment of the present invention, the compositions further contain sustained release components, for example, polymers (in the form for example of gels and microspheres), such as poly (D,L,-lactide) or poly(D,L-lactide co-glycolide), in amounts effective to reduce local diffusion rates and/or corticosteroid particle dissolution rates. The result is a flatter elimination rate profile with a lower Cₘₐₓ and a more prolonged therapeutic window, thereby extending the time between required injections for many patients.

Any suitable, preferably conditionally acceptable, release component may be employed. Useful examples are set forth above. The sustained release component is preferably biodegradable or bioabsorbable in the eye so that no residue remains over the long term. The amount of the delayed release component included may very over a relatively wide range depending, for example, on the specific sustained release component is being employed, the specific release profile desired and the like factors. Typical amounts of delayed release components, if any, included in the present compositions are in a range of about 0.05 to 0.1 to about 0.5 or about 1 or more percent (w/v) (weight of the ingredient in the total volume of the composition) of the composition.

The present compositions can be prepared using suitable blending/processing techniques or techniques, for example, one or more conventional blending techniques. The preparation processing should be chosen to provide the present compositions in forms which are useful for placement or injection into the posterior segments of eyes of humans or animals. In one useful embodiment a concentration corticosteroid component dispersion is made by combining the corticosteroid component with water, and the excipient (other than the viscosity inducing component) to be included in the final composition. The ingredients are mixed to disperse the corticosteroid component and then autoclaved. Alternatively, the steroid powder may be γ-irradiated before addition to the sterile carrier. The viscosity inducing component may be purchased sterile or sterilized by conventional processing, for example, by filtering a dilute solution followed by lyophylization to yield a sterile powder. The sterile viscosity inducing component is combined with water to make an aqueous concentrate. Under aseptic conditions, the concentrated corticosteroid component dispersion can be blended or mixed and added or combined as a slurry to the viscosity inducing component concentrate. Water is added in a quantity sufficient (q.s.) to provide the desired composition and the composition is mixed until homogenous.

Methods of using the present composition are provided and are included within the scope of the present invention. In general, such methods comprise administering a composition in accordance with the present invention to a posterior segment of an eye of a human or animal, thereby obtaining a desired therapeutic effect. The administering step advantageously comprises at least one of intravitreal injecting, subconjunctival injecting, sub-tenon injecting, retrobulbar injecting, suprachoroidal injecting and the like. A syringe apparatus including an appropriately sized needle, for example, a 27 gauge needle or a 30 gauge needle, can be effectively used to inject the composition with the posterior segment of an eye of a human or animal.

Ocular conditions which can be treated or addressed in accordance with the present invention include, without limitation, the following:

Maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like. The present methods may comprise a single injection into the posterior segment of an eye or may involve repeated injections, for example over periods of time ranging from about one week or about 1 month or about 3 months to about 6 months or about 1 year or longer.

### EXAMPLES

The following non-limiting Examples are presented to exemplify aspects of the present invention.

### Examples 1 to 4

Four compositions are as follows:

TABLE 1

| Ingredient | Example 1 | Example 2 | Example 3 | Example 4 |
|---|---|---|---|---|
| Triamcinolone | 2% | 2% | 4% | 4% |
| acetonide | (w/v) | (w/v) | (w/v) | (w/v) |
| Sodium Hyaluronate | 0.05% | 0.5% | 0.05% | 0.5% |
| (0.6 × 10⁶ | (w/v) | (w/v) | (w/v) | (w/v) |
| DALTONS) | | | | |
| Sodium Phosphate | 0.4% | 0.4% | 0.4% | 0.4% |
| | (w/v) | (w/v) | (w/v) | (w/v) |
| Vitamin E-TPGS | 0.5% | 0.5% | 0.0 | 0.0 |
| | (w/v) | (w/v) | | |
| γ-cyclodextrin | 0.5% | 0.5% | 0.0 | 0.0 |
| | (w/v) | (w/v) | | |
| Water for Injection | q.s. | q.s. | q.s. | q.s. |
| Viscosity at shear rate | 20 cps | 500 cps | 20 cps | 500 cps |
| 0.1/second | | | | |

Each of these compositions is prepared as follows.

A concentrated triamcinolone acetonide dispersion is made by combining triamcinolone acetonide with water, Vitamin E-TPGS and y-cyclodextrin, if any. These ingredients are mixed to disperse the triamcinolone acetonide, and then autoclaved. The sodium hyaluronate may be purchased as a sterile powder or sterilized by filtering a dilute solution followed by lyophylization to yield a sterile powder. The sterile sodium hyaluronate is dissolved in water to make an aqueous concentrate. The concentrated triamcinolone acetonide dispersion is mixed and added as a slurry to the sodium hyaluronate concentrate. Water is added q.s. (quantum sufficit, as much as suffices, in this case as much as is required to prepare the homogenous mixture, dispersion, gel or suspension) and the mixture is mixed until homogenous.

Each of these compositions produced a loose flocculation of triamcinolone acetonide that is easily re-suspended by gentle inversion. These compositions can be marketed in small volume pharmaceutical grade glass bottles, and are found to be therapeutically effective against macular edema when injected intravitreally into human eyes.

### Examples 5 to 7

Three compositions are as follows:

TABLE 2

| Ingredient | Example 5 | Example 6 | Example 7 |
|---|---|---|---|
| Triamcinolone acetonide | 2.0% (w/v) | 4.0% (w/v) | 8.0% (w/v) |
| Sodium hyaluronate | 3.0% (w/v) | 2.5% (w/v) | 2.0% (w/v) |
| Sodium Phosphate | 0.4% (w/v) | 0.4% (w/v) | 0.4% (w/v) |
| Water for Injection | q.s. | q.s. | q.s. |
| Viscosity at shear rate | 300,000 cps | 180,000 cps | 100,000 cps |
| 0.1/second | | | |

These compositions are prepared in a manner substantially analogous to that set forth in Example 1.

The high viscosities of the compositions substantially slows the particle sedimentation rate to an extent that no resuspension processing is necessary or required over the estimated shelf life, e.g., about 2 years, of the compositions. These compositions can be marketed in prefilled syringes since they can not easily be removed by a needle and syringe from a container. However, with the compositions in prefilled syringes, the compositions can be effectively injected into the posterior segment of an eye of a human using a 27 gauge or a 30 gauge needle to provide a desired therapeutic effect in the human eye.

The compositions of Examples 5 to 7 employ or contain a sufficient concentration of high molecular weight sodium hyaluronate so as to form a gelatinous plug or drug depot upon intravitreal injection into a human eye. Triamcinolone acetonide particles are, in effect, trapped or held within this viscous plug, so that undesirable pluming does not occur, and the risk of drug particles disadvantageously settling directly on the retinal tissue is substantially reduced, for example, relative to using a composition with a water-like viscosity, such as Kenalog® 40. Since sodium hyaluronate solutions are subject to dramatic shear thinning, these formulations are easily injected through 27 gauge or even 30 gauge needles.

### Examples 8 and 9

Two compositions are as follows:

TABLE 3

| Ingredient | Example 8 | Example 9 |
|---|---|---|
| Triamcinolone acetonide | 2.0% (w/v) | 8.0% (w/v) |
| Sodium hyaluronate | 2.5% (w/v) | 2.3% (w/v) |
| (polymeric) | | |
| Sodium chloride | 0.63% (w/v) | 0.63% (w/v) |
| dibasic sodium | 0.30% (w/v) | 0.30% (w/v) |
| phosphate, heptahydrate | | |
| Monobasic sodium | 0.04% (w/v) | 0.04% (w/v) |
| phosphate, monohydrate | | |
| Water for Injection | q.s. | q.s. |
| Viscosity at shear rate | 170,000 ± 25% cps | 200,000 ± 25% cps |
| 0.1/second | | |

These compositions are prepared in a manner substantially analogous to that set forth in Example 1.

The high viscosities of the compositions substantially slows the particle sedimentation rate to an extent that no resuspension processing is necessary or required over the estimated shelf life, e.g., about 2 years, of the compositions. These compositions can be marketed in prefilled syringes since they can not easily be removed by a needle and syringe from a container. However, with the compositions in prefilled syringes, the compositions can be effectively injected into the posterior segment of an eye of a human using a 27 gauge or a 30 gauge needle to provide a desired therapeutic effect in the human eye.

The sodium hyaluronate powders used in these compositions (as well as in the other compositions identified in the Examples herein) have water contents in a range of about 4% to about 20%, preferably about 4% to about 8%, by weight. Differences in the average molecular weight of the hyaluronate used can result in variation in the viscosity of compositions in accordance with the present invention which have the same nominal chemical make-ups. Thus, the viscosities indicated herein should be understood to be target viscosities, with the composition being acceptable for use if the actual viscosity of the composition is within plus or minus (±) about 25% or about 30% or about 35% of the target viscosity.

Because each of the compositions set forth in the Examples has a density of about 1 gm/ml, the percentages set forth herein as being based on weight per volume (w/v) can also be considered as being based on weight per weight (w/w).

The compositions of Examples 8 and 9 employ or contain a sufficient concentration of high molecular weight (i.e. polymeric) sodium hyaluronate so as to form a gelatinous plug or drug depot upon intravitreal injection into a human eye. Preferably the average molecular weight of the hyaluronate used is less than about 2 million, and more preferably the average molecular weight of the hyaluronate used is between about 1.3 million and 1.6 million. The triamcinolone acetonide particles are, in effect, trapped or held within this viscous plug of hyaluronate, so that undesirable pluming does not occur upon intravitreal injection of the formulation. Thus, the risk of drug particles disadvantageously settling directly on the retinal tissue is substantially reduced, for example, relative to using a composition with a water-like viscosity, such as Kenalog® 40. Since sodium hyaluronate solutions are subject to dramatic shear thinning, these formulations are easily injected through 27 gauge or even 30 gauge needles.

The triamcinolone acetonide used in the formulations set forth herein has the chemical name 9-Fluoro-1 1,21 -dihydroxy-1 6,17-[1 -methylethylidenebis(oxy)]pregna-1,4-diene-3,20-dione, and can have the following structure

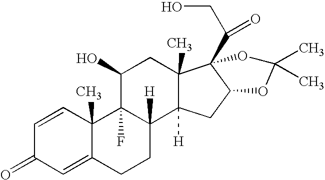

The molecular formula of triamcinolone acetonide is C₂₄H₃₁FO₆ and its molecular weight is 434.49. The solubility of triamcinolone acetonide in water is about 25.4 μL/mL.

The Examples 8 and 9 formulations are prepared as sterile products of a uniform, opaque white dispersion of microfine triamcinolone acetonide particles suspended in a hyaluronate-based polymeric hydrogel, intended for intravitreal injection.

The Examples 8 and 9 formulations can be used top treat, for example, macular edema in patients with diabetes, central retinal vein occlusion, and branch retinal vein occlusion. Notable the Examples 8 and 9 formulations are formulated using only excipients that are fully compatible (i.e. non-toxic) to the eye, particularly to the retina. The Examples 8 and 9 formulations (2% (w/w) and 8% (w/w) triamcinolone acetonide, respectively) are buffered at physiological pH with a low concentration of sodium phosphate salts; rendered isotonic with sodium chloride, and use Water for Injection, USP, as the vehicle.

A target dosage of 1 mg of the triamcinolone acetonide active agent can be delivered in a 50 mg (approximately 48 μL) injection of the Example 8 2% (w/w) triamcinolone acetonide gel suspension formulation. A target dosage of 4 mg of the triamcinolone acetonide active agent can be delivered in a 50 mg (approximately 48 μL) injection of the Example 9 8% (w/w) triamcinolone acetonide gel suspension formulations.

As noted, the triamcinolone present in our formulations does not rapidly, or even slowly, settle out from or precipitate from the formulations. Significantly our Example 8 and 9 formulations have a shelf life of at least two years, meaning that these formulations can be left standing (without agitation) for up to about two years before administration to an eye, and after two years the same formulations can still provide a consistent and accurate dose of triamcinolone upon injection to the formulation to an eye. For example, upon preparation (as set froth in Example 15), 50 μL of our 8% formulation provides 4 mg of triamcinolone acetonide, and if left standing for up to about 2 years 50 μL of our 8% formulation stills provides 4 mg±15% of triamcinolone acetonide, thereby meeting the U.S.P. definition of consistent dosage after storage.

As noted, the composition of triamcinolone 2% injectable gel suspension (Example 8) is triamcinolone 2.0% (w/w), sodium hyaluronate, sodium chloride, dibasic sodium phosphate (heptahydrate), monobasic sodium phosphate (monohydrate), and water for injection.). The composition of triamcinolone 8% injectable gel suspension (Example 9) is triamcinolone 8.0% (w/w), sodium hyaluronate, sodium chloride, dibasic sodium phosphate (heptahydrate), monobasic sodium phosphate (monohydrate), and water for injection.

The triamcinolone acetonide injectable gel suspension we have invented is a Docket 17642C[P2 viscous suspension of triamcinolone acetonide formulated at concentrations of 8% and 2% with sodium hyaluronate, sodium chloride, dibasic sodium phosphate (heptahydrate), monobasic sodium phosphate (monohydrate), and water for injection (i.e. the formulations of Examples 8 and 9 respectively). The suspensions are prepared to have physiologic pH, and to be isotonic, and preservative-free. The Examples 8 and 9 suspensions can be supplied in single-use glass syringes with fixed 27 gauge needles. The syringes are overfilled to 0.17-0.18 mL, and calibrated to deliver 0.05 mL when primed to a black or blue mark on the barrel of the syringe to thereby provide the 2% and 8% suspensions to deliver 1 mg and 4 mg of triamcinolone, respectively (the pre-filled syringes are made by Allergan, Inc., Irvine, California).

### Example 10

### Ocular and Systemic Pharmacokinetics of a 4% (4 mg) Triamcinolone Acetonide Gel Suspension Formulation upon Intravitreal Injection

An experiment was carried out to evaluate the intraocular and systemic pharmacokinetics of triamcinolone acetonide gel suspensions (TAA_{gs}) following intravitreal administration. The formulations used were: (1) 4% w/v (40 mg/mL) triamcinolone acetonide formulated as a high viscosity borate-buffered 2.5% (w/w) hyaluronic acid suspension, and; (2) 16% w/v (160 mg/mL) triamcinolone acetonide formulated as a high viscosity borate-buffered 2.5% (w/w). 100 μL of each formulation was injected into separate rabbit eyes using a 25-gauge needle syringe to provide 4 mg or 16 mg of triamcinolone actinide, respectively. Except as noted the formulations used in this Example 10 were the same as the Example 8 and 9 formulations. For example, the same type of sodium hyaluronate was used in these Example 10 formulations.

Following a single intravitreal injection (New Zealand albino rabbits) of 100 μL of the 4% w/v triamcinolone acetonide formulation (4 mg triamcinolone acetonide), aqueous humor, vitreous humor, retina and plasma were collected on days 1, 3, 10, 17, 31 and 45 and analyzed for triamcinolone acetonide by liquid chromatography tandem mass spectrometry. In vitreous humor, the maximal triamcinolone acetonide concentration (Cₘₐₓ) was 385 μg/g on Day 10. The relatively constant concentrations (i.e. sustained release) of triamcinolone acetonide were observed from Day 1 to Day 45. (Tables 4 and 5) Therefore, the TAA_{gs} formulation delivers a relatively stable concentration (i.e. approximately zero-order release kinetics) of triamcinolone acetonide to the retina over at least 45-day period following single intravitreal injection.

Table 4 also shows that the ratio of the amount of triamcinolone acetonide present in the vitreous to the amount of triamcinolone acetonide present in the aqueous chamber can be greater than 1000:1 at all time points.

Table 5 shows that ratio of the total amount of triamcinolone acetonide present in the vitreous over the 45 day study period to the total amount of triamcinolone acetonide present in the aqueous humor over the 45 day study period can be greater than about 5,000:1.

TABLE 4
Triamcinolone acetonide concentration in aqueous humor, retina and
vitreous humor after a single intravitreal injection of a 4% triamcinolone
acetonide formulation in albino rabbits

Triamcinolone Acetonide
(μg/mL or μg/g)

| Timepoint (Day) | Aqueous Humor | Vitreous Humor |
|---|---|---|
| 1 | 0.319 | 382 |
| 3 | 0.200 | 335 |
| 10 | 0.052 | 385 |
| 17 | 0.033 | 338 |
| 31 | 0.014 | 185 |
| 45 | 0.011 | 222 |

TABLE 5
Pharmacokinetic parameters of triamcinolone acetonide in ocular tissues
after a single intravitreal injection of 4% triamcinolone acetonide
formulation in albino rabbits

| | Cₘₐₓ | AUC_{0-45day} |
|---|---|---|
| Treatment | (μg/mL or μg/g) | (μg · day/mL or μg · day/g) |
| Vitreous Humor | | |
| 4% Triamcinolone | 385 | 12500 |
| Acetonide Injectable | | |
| Aqueous Humor | | |
| 4% Triamcinolone | 0.319 | 2.36 |
| Acetonide Injectable | | |

Following intravitreal administration of the 4% TAA_{gs} formulation in albino rabbits, triamcinolone acetonide was absorbed into the systemic circulation with mean plasma Cₘₐₓ of 15.8 ng/mL at 1 day postdose. Between days 2 and 45, plasma levels drop to 7 and 1 ng/mL, respectively. Thus, our the triamcinolone acetonide gel suspension formulations are free of excipients with known ocular toxicity, and through sustained release from the gel delivers prolonged levels of triamcinolone acetonide to the vitreous and retina.

### Example 11

### Triamcinolone Gel Suspensions to Treat Ocular Conditions

Introduction

As set forth herein, we have invented triamcinolone acetonide gel suspensions (TAA_{gs}) and their use to treat various ocular conditions, including macular edema, such as macular edema associated with diabetes and/or a retinal vein occlusion, (branch or central), and to maintain or to improve visual acuity. Our TAA_{gs} formulations can contain a polymeric hyaluronic acid.

The blood-aqueous barrier (“BAB”) is a membrane of the capillary bed of the ciliary body of the eye that influences or controls two-way transfer of fluids between the aqueous chamber of the eye and the blood. The BAB acts as an anatomical mechanism to reduce or prevent exchange of materials between the chambers of the eye and the blood.

The blood-retinal barrier (“BRB”) is composed of specialized nonfenestrated tightly-joined (tight junction) retinal epithelium and adjacent retinal blood vessel endothelium cells that forming a transport barrier for certain substances between the retinal capillaries and the retinal tissue. BRB breakdown is symptomatic of various retinal ocular conditions, including reduced visual acuity, macular edema, macula degeneration, retinal swelling, and other retinopathies, including diabetic retinopathy.

This experiment was designed to assess efficacy and duration of action of particular TAA_{gs} polymeric hyaluronic acid formulations injected intravitreally to treat break down (deterioration) of the blood-aqueous barrier (“BAB”) and of the blood-retinal barrier (“BRB”) in mammal eyes. Generally, a reduced BRB breakdown is a desirable condition or state, as it indicates a stabilized or more normal or more healthy retina (tightened barrier). On the other hand, where in a model system a BAB breakdown is induced, it is considered a positive or beneficial result if upon intravitreal administration of a steroid, such as a corticosteroid or an anti-inflammatory steroid into an eye with BAB breakdown, an improvement of the BAB breakdown is not observed. Failure of BAB breakdown to be reduced or repaired is an indication that the steroid intravitreally administered has not in significant quantity made it's way (i.e. by diffusion and/or by an active transport mechanism) to the aqueous (or anterior) chamber (“AC”) of the eye. It is known that AC presence of various steroids can cause increased intraocular (i.e. aqueous humor) pressure (elevated IOP is symptomatic of glaucoma) and/or cataract generation.

Summary

The experiment was carried out using intravitreal injection of either a 1 mg or 4 mg triamcinolone acetonide gel suspension (TAA_{gs}) (the Example 8 and 9 formulations, respectively) in a rabbit model of VEGF-mediated blood-aqueous barrier (BAB) and blood-retinal barrier (BRB) breakdown. The model system used is set forth in Edelman et al., Corticosteroids inhibit VEGF-induced vascular leakage in a rabbit model of blood-retinal and blood-aqueous barrier breakdown, Exp Eye Res February 2005;80(2):249-58, although instead of intravitreal injection of a triamcinolone acetonide saline suspension, the formulation of Examples 8 and 9 above were used in this experiment.

BAB breakdown was measured by anterior chamber fluorophotometry. BRB breakdown was measured by vitreal fluorophotometry and subjective grading of fluorescein angiograms. In addition, VEGF-induced changes in vessel caliber and tortuosity (AVC-T) were assessed by subjective scoring of fundus images. The equipment and procedures used to obtain anterior chamber fluorophotometry, vitreal fluorophotometry, fluorescein angiograms, and fundus images were as set forth in Edelman (2005) supra.

The results obtained in this experiment can be summarized as follows:

1. intravitreal 1 mg TAA_{gs} had no effect on VEGF-induced BAB at any time point as compared to control eyes.

2. intravitreal 1 mg TAA_{gs} significantly inhibited VEGF-induced BRB and AVC-T through at least 6 weeks.

3. intravitreal 4 mg TAA_{gs} did not significantly inhibit VEGF-induced BAB breakdown at 10, 22, and 30 weeks.

4. intravitreal 4 mg TAA_{gs} significantly blocked VEGF-induced angiographic BRB breakdown for at least about 14 weeks, fluorophotometric BRB breakdown for at least about 22 weeks, and ΔVC-T for at least about 14 weeks, and in at least some rabbit eyes for up to at least about 30 weeks.

Methods

Female Dutch Belt rabbits (5 to 6 months old) were randomly assigned to a no treatment group (control; n=12 eyes), to a group to receive intravitreal injection of 1 mg TAA_{gs} (n=8 eyes), or to a group to receive intravitreal injection of 4 mg TAA_{gs} (n=10 eyes). 50 μL of the 2 % or 8% (Example 8 and Example 9 formulations respectively) TAA_{gs} formulations were intravitreally injected into eyes of the later two groups on Day 1. Since the VEGF responses are transient and return to baseline by one week (See Edelman et al.(2005), supra), the TAA_{gs} duration of action was determined by injecting VEGF intravitreally at multiple times points over a 10 week for 1 mg TAA_{gs} and over a 30 week for the 4 mg TAA_{gs} Thus, the VEGF was injected intravitreally at 2 weeks, 6 weeks and 10 weeks after study initiation for the 1 mg TAA_{gs} study rabbits The VEGF was injected intravitreally at 2 weeks, 6 weeks and 10 weeks, 14 weeks, 22 weeks, and 30 weeks after study initiation for the 4 mg TAA_{gs} study rabbits.

Drug (TAA_{gs}) Injection

General anesthesia was initiated by isoflurane inhalation and the ocular surface was anesthetized with 1-2 drops of 1% proparacaine. Rabbits were then placed on a heated pad, covered with a sterile drape, and both eyes were treated with Betadine for 30 seconds and rinsed with sterile saline. 50 μL of the 1 mg or 4 mg TAA_{gs} formulations (the Example 8 and 9 formulations, respectively) were administered via their original glass syringes and 27 gauge needle, and each syringe was calibrated to the blue line prior to injection). The syringe needle was inserted about 3 mm posterior to the limbus and aimed inferior and posterior. After injection, the needle was removed and the eye was checked for leakage.

Rabbit Model of VEGF-Induced Vasculopathies

The Edelman (2005) A model of BRB and BAB breakdown was used to determine the pharmacologic duration of action after intravitreal injection of 1 and 4 mg TAA_{gs}. Rabbits were placed on a heated pad, covered with a sterile drape, and 500 ng of recombinant human vascular endothelial growth factor (165 amino acid variant; VEGF₁₆₅, obtained from R & D Systems, Minneapolis, Minn.) in 50 μL sterile phosphate buffered saline was injected intravitreally into all eyes via a 27G needle.

Forty-Eight hours after VEGF injection, eyes were dilated with 10% phenylephrine HCl and 1% cyclopentolate HCI. Anesthesia was induced via subcutaneous injection of 50 mg/kg ketamine and 10 mg/kg xylazine. Once anesthetized, the rabbit fundus was visualized with a Zeiss retinal camera and fundus images were obtained and stored on a personal computer. Sodium fluorescein was administered intravenously (11.75 mg/kg) and late phase angiograms were obtained after 5-10 min. Fifty minutes after fluorescein injection BRB and BAB integrity were measured using scanning ocular fluorophotometry (Fluorotron Master).

Fundus images were graded on a scale of 1 (normal) to 5 (maximal blood vessel dilation and tortuosity) by three masked observers. Retinal fluorescein leakage was scored from angiograms read by masked observers on a scale of 1 (no fluorescein leakage =normal) to 5 (maximum fluorescein leakage).

Angiogram and fundus image scores were compared with an unpaired non-parametric Wilcoxon Rank Sum/Mann-Whitney U-Test. Fluorophotometric measurements (area under the curve) were analyzed with a two tailed Students t-test. P-values ≦0.05 are determined to be significant.

Results

2 % TAA_{gs}: 1 mg Dose

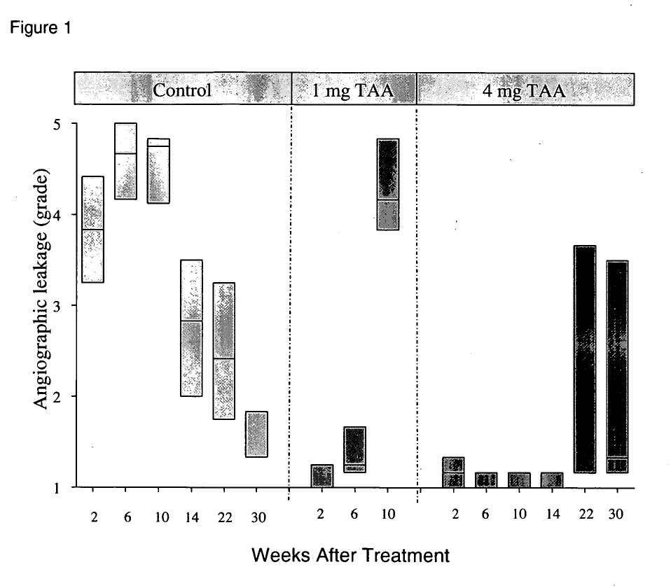

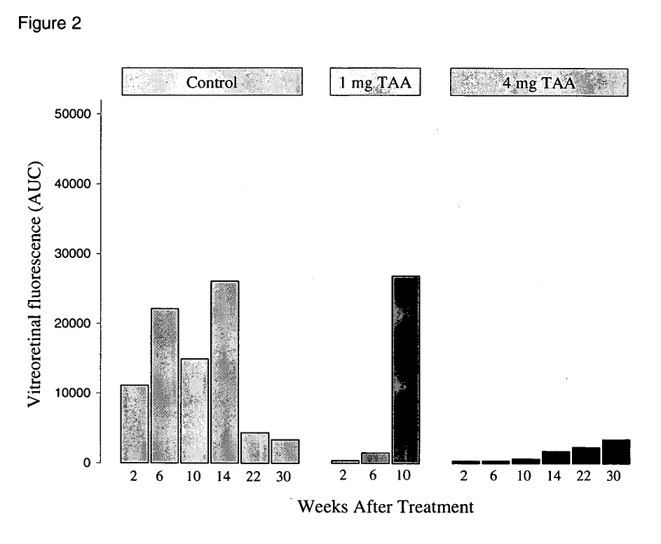

1. Effect on blood-retinal barrier (BRB) breakdown. Subjective grading of angiograms (FIG. 1) or vitreal fluorophotometry (FIG. 2) shows that VEGF-induced BRB breakdown was suppressed in eyes treated with 1 mg TAA_{gs} through at least about six weeks.

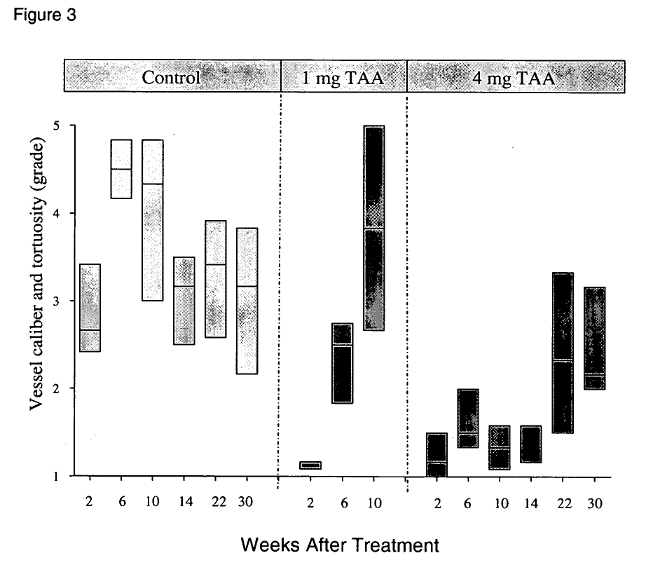

2. Effect on changes in retinal vessel caliber-tortuosity (ΔVC-T). Subjective grading of fundus images (FIG. 3) shows that VEGF-induced ΔVC-T was suppressed in eyes treated with 1 mg TAA_{gs} through at least about six weeks.

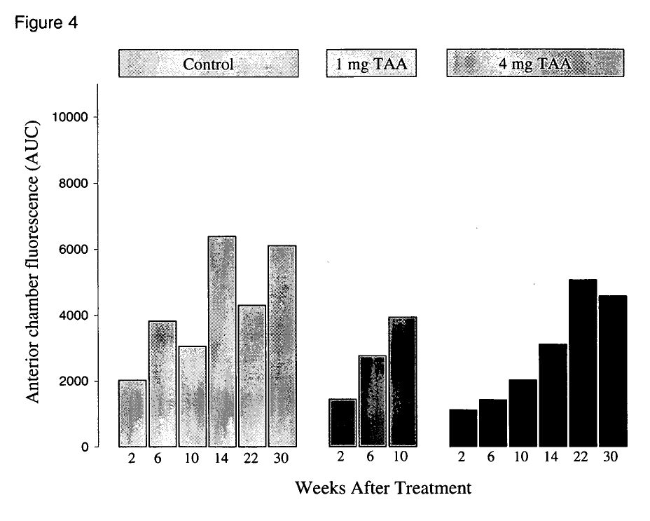

3. Effect on blood-aqueous barrier (BAB) breakdown. Anterior chamber fluorophotometry shows that the extent of VEGF-induced BAB breakdown was not suppressed in rabbit eyes treated with the 1 mg TAA_{gs} for at least at about 10 weeks (FIG. 4).

8 % TAAgs: 4 mg Dose

1. Effect on BRB breakdown. Subjective grading of angiograms (FIG. 1) or vitreal fluorophotometry (FIG. 2) shows that VEGF-induced BRB breakdown was suppressed in eyes treated with 4 mg TAA_{gs} for between about fourteen weeks (FIG. 1) and twenty weeks (FIG. 2).

2. Effect on ΔVC-T Subjective grading of fundus images (FIG. 3) shows that VEGF-induced ΔVC-T was clearly suppressed in all eyes treated with 4 mg TAA_{gs} for at least fourteen weeks, and for some rabbits through twenty two to thirty weeks (210 days or about 7.5 months).

3. Effect on BAB breakdown. Anterior chamber fluorophotometry (FIG. 4) shows that the extent of VEGF-induced BAB breakdown was not significantly suppressed at 10, 22, and 30 weeks.

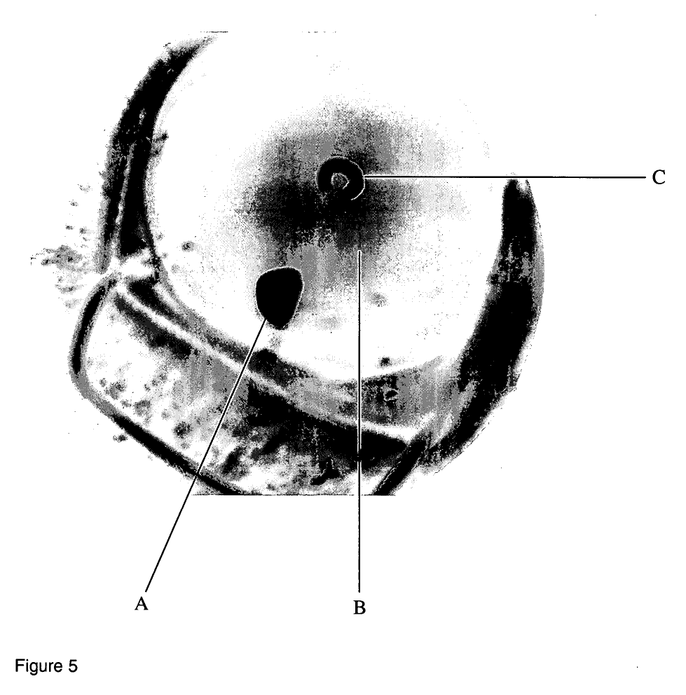

These results showed significant inhibition of VEGF-induced BRB responses for at least six weeks with intravitreal 1 mg TAA_{gs} (FIGS. 1-3),and for at least 30 weeks with intravitreal 4 mg TAA_{gs} (FIG. 3). Note that FIG. 5 (a negative image of a photograph of the eye of a rabbit in this Example 11 thirty weeks after intravitreal injection of 50 μL of the 4 mg TAA_{gs} formulation) shows that our TAA_{gs}. formulation can remain intact in the vitreous for a prolonged period. In FIG. 5 item A is the intact, single object (bolus) intravitreal 4 mg, 50 μL TAA gel suspension 30 weeks after intravitreal injection. B is the vitreous chamber and C is a light reflection artifact.

Thus, based upon a demonstrated therapeutic effect for as long as thirty weeks after intravitreal injection of a TAA_{gs} formulation, which TAA_{gs} which remains intact in the vitreous for the same period, our TAA_{gs} formulation can be characterized as a sustained release, biocompatible, biodegradable implant.

Thus, the results from this experiment demonstrate that intravitreal administration of a TAA_{gs} formulation can be used to treat a retinal disease or condition (such as a retinal disease or condition which includes BRB breakdown or deterioration) with no or little diffusion of drug to the anterior chamber (as determined for example by the Example 10 data, and by the lack of or of a reduced effect on BAB breakdown set forth in this Example 11). It can therefore be concluded that our TAA_{gs} formulations can be used to advantageously treat a retinal disease or condition with, for example, little or no IOP elevation (with reduced incidence of glaucoma therefore) and with no or little inducement of cataract formation or intraocular inflammation.

Our TAA_{gs} formulations have numerous novel and advantageous characteristics making them well suited for the treatment of ocular conditions, such as posterior ocular conditions, such as macular edema, such as diabetic macula edema. For example our TAA_{gs} formulation (for example the Examples 8 and 9 formulations) do not contain any preservatives or excipients such as an alcohol (such as a benzyl alcohol) or a polysorbate (such as a polysorbate 80). Thus our TAA_{gs} formulations have a reduced retinal toxicity.

Additionally, our TAA_{gs} formulations have superior depot and release characteristics. Intravitreal injection of an aqueous (i.e. in saline) solution of a triamcinolone provides active agent which quickly (in a matter of hours) diffuses out of the retina. Our TAA_{gs} formulations have a longer duration of intravitreal therapeutic activity because therapeutic amounts of the triamcinolone can diffuse out of the gel over a period of thirty weeks or more. Thus, use of a suspending agent such as a polymeric hyaluronate can provide the consistency permitting substantially zero order kinetics release of the triamcinolone form the hyaluronate, proving thereby both an extended duration of effect of the triamcinolone and reduced levels and therefore a reduced effect of the triamcinolone upon the anterior chamber of the eye and a reduced systemic exposure to the active agent.

Our invention comprises triamcinolone acetonide injectable gel suspensions formulated viscous suspensions of triamcinolone acetonide at concentrations of, for example, 8% and 2% with sodium hyaluronate, sodium chloride, dibasic sodium phosphate (heptahydrate), monobasic sodium phosphate (monohydrate), and water for injection. The triamcinolone acetonide injectable gel suspensions are preferably at physiologic pH, isotonic, and preservative-free. Triamcinolone acetonide injectable gel suspensions within the scope of our invention can be supplied in single-use glass syringes with fixed 27 gauge needles. The syringes can be overfilled to 0.17-0.18 mL, and calibrated to deliver 0.05 mL when primed to a black mark on the barrel of the syringe to thereby deliver, for example, 2% and 8% suspensions of 1 mg and 4 mg of triamcinolone, respectively. Our triamcinolone acetonide injectable gel suspensions can be defined as implants which upon injection (i.e. implantation) into the vitreous provided sustained release (i.e. over a period of up to seven months or longer) from the compact gel bolus injected.

Our triamcinolone acetonide injectable gel suspensions are preferably not used as visualizing agents, for example in conjunction with a vitrectomy (see eg U.S. Pat. No. 6,395,294) because the viscous, gel nature of our formulations prevents them for rapidly spreading out within the vitreous, as is required for a vitreal visualization agent (such as for example triptan vision blue, or water or saline based triamcinolone solutions or formulation, such as Kenalog®). A lower molecular weight hyaluronate with triamcinolone acetonide can be used for visualization (for example with a viscosity at a shear rate of about 0.1/second of less than about 90,000 cps, such as for example about 1,000 to 10,000 cps), whereas the higher molecular weight hyaluronate of Examples 8 and 9 are preferred for use as in situ forming vitreous implants.

### Example 12

### Treatment of Macular Edema with Intravitreal Triamcinolone Acetonide Suspension

A 64 year old obese female patient with symptoms of diabetes presents with vision loss due to macula edema with central retinal vein occlusion and/or branch retinal vein occlusion. She receives intravitreal injection of 4 mg of a high viscosity triamcinolone acetonide (polymeric hyaluronate based) suspension, such as the Example 9 formulation.

Twelve months after injection she demonstrates an improved best corrected visual acuity of fifteen or more letters from baseline as determined using the Early Treatment of Diabetic Retinopathy Study (ETDRS) visual acuity chart.

### Example 13

### Treatment of a Posterior Ocular Condition with Intravitreal Triamcinolone Acetonide Suspension

Patients with a posterior ocular condition (such as a macular edema, uveitis, or macular degeneration) can be treated by intravitreal injection of 1 mg or 4 mg of a high viscosity triamcinolone acetonide gel (polymeric hyaluronate based) suspension, such as the Example 8 or Example 9 formulation. Alternately, the formulation can be administered by subconjunctival injection to treat the posterior ocular condition. These patients can demonstrate twelve months after injection an improved best corrected visual acuity of fifteen or more letters from baseline as determined using the Early Treatment of Diabetic Retinopathy Study (ETDRS) visual acuity chart.

To date in clinical studies being carried out, participated in, or supervised by the inventors or their colleagues over seven hundred patients have received intravitreal injection of the Example 8 or Example 9 formulations. Yet the incidence of aseptic endophthalmitis in these numerous patients has been 0%. This is striking when one notes that the incidence of endophthalmitis upon intravitreal injection of Kenalog is about 1% to 2%.

Thus, it is important to note that the desired therapeutic result (maintained or improved vision) can be obtained with little or no incidence of intraocular inflammation. Without wishing to be bound by theory we can postulate reasons for this exceptional result. Macrophages are involved with the removal of particulate material from the body through phagocytosis. However, particles of large morphology and irregular geometry can be toxic to macrophages and lead to cell death. The death of macrophages can lead to release of pro-inflammatory cytokines that cause both acute and chronic inflammation. Clinical examples of toxicity from particles include gouty arthritis, where urate crystals that range from 5 to 20 microns cause a debilitating arthritis. Helliwell P., Use of an objective measure of articular stiffness to record changes in finger joints after intra-articular injection of corticosteroid, Ann Rheum Dis 1997;56:71-73. Macrophages are injured when phagocytosing the urate crystals and this initiates the inflammatory response. When patients are treated with medication that reduces macrophage activity, such as colchicine, this leads to a dramatic improvement in the arthritis. Another example of joint inflammation related to particles is ‘crystal-induced synovitis’, where 1-2% of patients that receive intra-articular injections of Lederspan, Kenalog, or other corticosteroid depot formulation, develop a post-injection exacerbation of the joint inflammation. McCarty D., et al., Inflammatory reaction after intrasynovial injection of microcrystalline adrenocorticosteroid esters, Arthritis and Rheumatism, 7(4); 359-367 (1964). The particles in these formulations, which are on the average over 10 microns and have irregular morphology, are very similar to the urate crystals in joint of patients with gout. Significantly, in our formulations the triamcinolone particles (crystals) are not available to and/or are substantially ignored by macrophages due to the aggregation (suspension) of the triamcinolone particles in the high molecular weight hyaluronate used in our formulations. The fact that our triamcinolone formulations are in situ forming implants can also limit the exposure of whole or individual triamcinolone crystals to sensitive ocular tissues, concomitantly thereby limiting macrophage activation and hence also limiting or preventing an intraocular inflammatory response. It is important to note that with our formulation the particular high viscosity hyaluronic acid polymer chosen maintains the triamcinolone crystals in a collective matrix that acts as a sustained-release reservoir which decrease the need for frequent repeat injections. Thus, our formulation forms a cohesive agglomerate upon intravitreal injection. The reduced surface area of such an agglomerate facilitates provision and maintenance of a lower release rate of the triamcinolone, as compared to much larger surface area saline suspension of a triamcinolone (such as Kenalog). The cohesiveness of our formulation is exemplified by the fact that the formulation maintains its internal consistency (i.e. its shape after injection) for at least about 30 weeks after intravitreal injection (see FIG. 5).

Additionally, the compositions of our invention are preferably formulated with hyaluronic acid, a material known for its anti-inflammatory abilities. Dea I. et al., Hyaluronic acid: a novel, double helical molecule, Science, Feb. 9, 1973; 179(73):560-2.).

Furthermore, the absence of preservatives and/or stabilizers (such as benzyl alcohol and polysorbate 80) in our formulation reduces the retinal toxicity of our formulations as compared to formulations which contain one or more preservatives and/or stabilizers

The combination of these five factors (lack of injury to macrophages, low availability of the triamcinolone crystal to macrophages, use of a biocompatible polymer, use of a high viscosity biocompatible polymer, and absence of preservatives and stabilizers provides an optimal ophthalmic delivery system which limits the incidence of post-injection aseptic endophthalmitis.

A preferred embodiment of our invention can be the Example 8 and 9 formulations in which the average diameter of the triamcinolone particles present in the formulations is less than 10 microns and preferably less than 5 microns, and additionally with a uniform (spherical) morphology. It has been shown in the pulmonary literature that micronized particles of corticosteroids, <10 microns, and preferably <5 microns, are less injurious to macrophages, and have the potential for less inflammation. (Robert A. Freitas Jr., Nanomedicine, Volume IIA: Biocompatibility, Landes Bioscience, Georgetown, Tex., 2003). Thus, preparing our formulations with a median triamcinolone particle size of <5 microns and with uniform shape provides formulation which are even more biocompatible in the vitreous and with less propensity to cause intraocular inflammation.

### Example 14

### Six Month Ocular and Systemic Pharmacokinetics of Triamcinolone Acetonide Following Intravitreal Injection of 2% (1 mg) and 8% (4 mg) Triamcinolone Acetonide Injectable Gel Suspension Formulations in Rabbit Eyes

An experiment was carried out to compare the ocular and systemic pharmacokinetics of triamcinolone acetonide (TA) following a single unilateral intravitreal injection of 2% (1 mg) and 8% (4 mg) TA injectable gel suspensions in new Zealand white rabbit eyes. These suspensions are the TA formulations of Examples 8 and 9, respectively.

Seventy-two female New Zealand White rabbits were obtained from Harlan (Indianapolis, Ind.). The rabbits were specific pathogen free (SPF), 17-18 weeks old and weighed 2.58-3.15 kg at the time of dosing. The seventy-two female rabbits were intravitreally injected with one of two TA doses (2% or 8%) and ocular and systemic pharmacokinetics monitored. Rabbits (four per group) were sacrificed on days 2, 4, 11, 32, 64, 92, 121, 151 and 183 for aqueous humor (AH), vitreous humor (VH) and plasma drug levels determined at each such time point at each of these three physiological locations. Samples were quantified using validated LC-MS/MS methods with assay range for TA of 0.2-20 ng/mL in plasma, 1-500 ng/mL in AH and 0.4-100 μg/mL in VH.

This study was a single treatment, parallel design, with 18 treatment groups and non-serial samples collected from each animal, as shown by Table 6.

TABLE 6
Study Design

| | Number | Treatment (Right Eye Only) | | Euthanasia and |
|---|---|---|---|---|
| | of | (Day 1 = Day of | TA | Necropsy |
| Group | Rabbits | intravitreal injection) | Dosed | (Day) |
| A | 4 | 2% (1 mg) Triamcinolone | 1 mg | 2 |
| | | Gel Suspension | | |
| B | 4 | 2% (1 mg) Triamcinolone | 1 mg | 4 |
| | | Gel Suspension | | |
| C | 4 | 2% (1 mg) Triamcinolone | 1 mg | 11 |
| | | Gel Suspension | | |
| D | 4 | 2% (1 mg) Triamcinolone | 1 mg | 32 |
| | | Gel Suspension | | |
| E | 4 | 2% (1 mg) Triamcinolone | 1 mg | 64 |
| | | Gel Suspension | | |
| F | 4 | 2% (1 mg) Triamcinolone | 1 mg | 92 |
| | | Gel Suspension | | |
| G | 4 | 2% (1 mg) Triamcinolone | 1 mg | 121 |
| | | Gel Suspension | | |
| H | 4 | 2% (1 mg) Triamcinolone | 1 mg | 151 |
| | | Gel Suspension | | |
| I | 4 | 2% (1 mg) Triamcinolone | 1 mg | 183 |
| | | Gel Suspension | | |
| J | 4 | 8% (4 mg) Triamcinolone | 4 mg | 2 |
| | | Gel Suspension | | |
| K | 4 | 8% (4 mg) Triamcinolone | 4 mg | 4 |
| | | Gel Suspension | | |
| L | 4 | 8% (4 mg) Triamcinolone | 4 mg | 11 |
| | | Gel Suspension | | |
| M | 4 | 8% (4 mg) Triamcinolone | 4 mg | 32 |
| | | Gel Suspension | | |
| N | 4 | 8% (4 mg) Triamcinolone | 4 mg | 64 |
| | | Gel Suspension | | |
| O | 4 | 8% (4 mg) Triamcinolone | 4 mg | 92 |
| | | Gel Suspension | | |
| P | 4 | 8% (4 mg) Triamcinolone | 4 mg | 121 |
| | | Gel Suspension | | |
| Q | 4 | 8% (4 mg) Triamcinolone | 4 mg | 151 |
| | | Gel Suspension | | |
| R | 4 | 8% (4 mg) Triamcinolone | 4 mg | 183 |
| | | Gel Suspension | | |

The seventy-two rabbits received a single unilateral (right eye) intravitreal injection of either 2% (1 mg) or 8% (4 mg) TA gel suspension. On day 1 each rabbit received an intravitreal injection into the midvitreous region through the dorsotemporal quadrant of the right eye, approximately 2-3 mm posterior to the limbus. For each injection, the needle of a pre-filled syringe (2% and 8% TA pre-filled syringes) was introduced through the dorsotemporal quadrant of the eye, approximately 2-3 mm posterior to the limbus, with the bevel of the needle directed downward and posteriorly to avoid the lens. 50 μL of either the 2% or 8% formulation was injected in a single bolus at a location roughly in the center of the vitreous.

There were no drug-related effects on body weight and mortality. Following a single intravitreal injection of either 2 or 8% TA gel suspension, TA was detected in the AH, VH and plasma at the earliest timepoint of Day 2. No contralateral diffusion of TA to the untreated eyes was detected in AH. The AH mean maximal concentrations (Cₘₐₓ) for 2% and 8% TA gel suspension were 27.6 ng/mL (Day 2) and 29.5 ng/mL (Day 11), respectively. The AH drug levels for the 2% and 8% dose were detectable up to Day 32 (4.15 ng/mL) and Day 151 (3.55 ng/mL), respectively. The area under the AH concentration time curve (AUC₀₋ₜₗₐₛₜ) was dose-dependent for the 2% (328 ng·day/mL) and 8% (1311 ng·day/mL) gel suspension with half-life (t_{1/2}) of 12.4 and 94.1 days, respectively.

Following intravitreal injection of 2% and 8% TA gel suspension, VH concentration of TA declined from 444 μg/g (57.6% dose remaining) at 2 days postdose to 22.1 μg/g (3.4% dose remaining) by 32 days post dose and 1460 μg/g (51.2% dose remaining) at 2 days to 33 μg/g (1.3% dose remaining) by 151 days post dose, respectively. No contralateral diffusion of TA to the untreated eyes was detected in VH at all timepoints except for the 8% dose on Day 2 (0.306 μg/g). The AUC₀₋ₜₗₐₛₜ for the 2% and 8% doses were 3410 μg·day/g and 68800 μg·day/g, respectively. The t_{1/2} for the 2% and 8% doses were 8.57 and 32.8 days, respectively. This 33 day half life is significantly greater than the 15 day half life reported for a saline suspension of TA (such as Kenalog) in the vitreous (Aubren (2004), supra). The 33 day half life can be expected to increase significantly to a half life of about 50-60 days in the VH of pathological and/or vitrectomized eyes.

The plasma Cₘₐₓ (Day 2) for the 2% and 8% doses were 4.12 ng/mL and 3.59 ng/mL, respectively. Plasma TA was detected for the 2 and 8% dose up to Day 11 and Day 64, respectively. The AUC₀₋ₜₗₐₛₜ for the 2% and 8% doses were 18.1 ng·day/mL and 83.6 ng·day/mL, respectively. The t_{1/2} for the 2% and 8% doses were 3.11 and 16.2 days, respectively.

Significantly (as noted above), TA was detected in the VH for the 2% (1 mg) and 8% (4 mg) TA gel suspension for up to 1 and 5 months postdose, respectively. The systemic exposure to TA following intravitreal injection was low and is expected to be relatively safe compared to systemic exposure of oral TA. Thus, it can be concluded that at least our 8% TA gel suspension can release TA into the vitreous over at least a 5 month (151 day) period, in the manner therefore of an in situ forming sustained release implant.

### Example 15

### Method for Making Injectable Triamcinolone Acetonide Gel Suspension Formulations

Preferred methods were developed for making the formulations of Examples 1 to 9.

The triamcinolone formulations are made as sterile, uniform, opaque white gel suspensions suitable for intraocular (such as intravitreal) injection. The manufacturing process involves two main stages: 1) sterile suspension bulk compounding and 2) aseptic filling. The bulk product manufacture includes preparations of three separate parts, followed by aseptic combination of these three parts. The aseptic filling operation is conducted in a class 100 environment, and the sterile bulk product may be filled into pre-sterilized ready-to-use syringes.

Micronized triamcinolone acetonide, USP, was purchased from Pfizer, Inc., Kalamazoo Mich. Typical and most useful particle sizes for this drug are 4-8 microns in diameter. Sodium hyaluronate powder was purchased from Hyaluron, Woburn, Mass. Typical and most useful molecular weights for this polymer are 1.0 to 1.9 million Daltons. When used, SBE7-β-cyclodextrin (Captisol®) was obtained from CyDex, Inc., Overland Park, Kans.

Part I is prepared in a main batch vessel that has capabilities of bulk heat sterilization and viscous fluid mixing. First, water for injection (WFI) at 40% of batch size is charged into the vessel and sodium chloride is dissolved. Triamcinolone powder is then added and dispersed with strong agitation. The suspension is heated and sterilized at above 121° C. for a sufficient time period by steam passing through the jacket of the vessel. After the bulk heat cycle is completed, the suspension is cooled down to room temperature.

Part II is prepared in an open vessel equipped with a top entering, variable speed mixer. First, WFI at 10% of batch size is charged into the vessel. Sodium phosphate salts and, optionally, a β-cyclodextrin derivative is added and dissolved. If necessary, the pH of the solution is adjusted with 1 N sodium hydroxide and/or 1 N hydrochloric acid. When a beta cyclodextrin is used in the formulation is can be dissolved along with the phosphate salts in this part II.

Part III is prepared in a Class 100 environment through a series of aseptic procedures. First, sodium hyaluronate is dissolved in WFI at dilute concentration, e.g., 0.2% w/w. The solution is sterile-filtered and sodium hyaluronate powder is recovered through bulk lyophilization. Finally, the sodium hyaluronate powder is reconstituted with sterile WFI at 50% of batch size.

Sterile bulk suspension is compounded by aseptically combining the three parts. First, Part II solution is filtered into sterile Part I in the main batch vessel using a 0.2 micron sterilizing grade filter. Part III is then aseptically transferred into the main batch vessel. Finally, the bulk is blended under low shear conditions to achieve uniformity. The final bulk suspension is held in a controlled area before aseptic filling.

Aseptic filling operations are performed in a Class 100 environment. Sterile bulk suspension is first filtered through a clarification screen into a sterile holding container. The bulk is then transferred to the filling machine and filled into pre-sterilized syringes. The filled units are transferred to the packaging area for application of tamper-evident seals, labeling and cartoning.

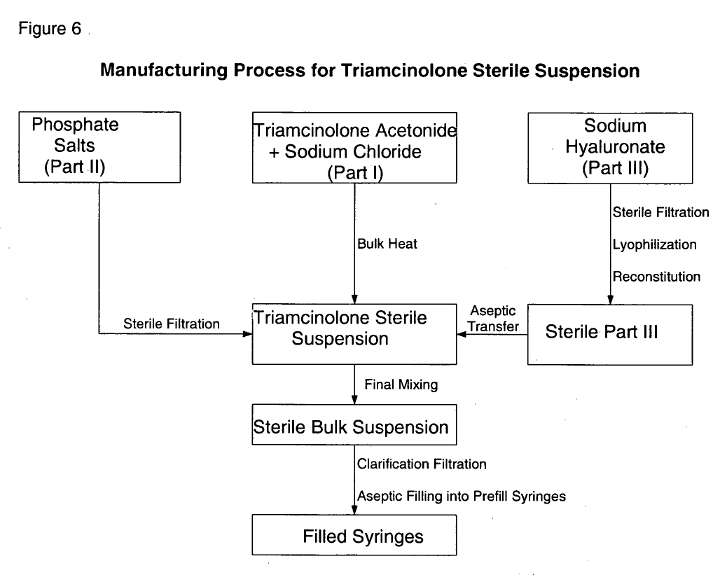

The pharmaceutical manufacturing process of this Example 15 for making triamcinolone sterile suspensions is illustrated by the FIG. 6 process flow chart.

Although not shown in FIG. 6, after Part III has been made (and before the lyophilization step is applied to Part III), Part III can be heated at between about 120° C. and about 130° C. for between about 25-35 minutes. Doing so both sterilizes the hyaluronate and can reduce the initial 1 million to 1.9 million Daltons molecular weight of the hyaluronate used in our formulation by about 20% to about 30% (i.e. to between about 0.7 million to about 1.3 million Daltons), thereby permitting use of a higher (i.e. 30 gauge) gauge injection needle.

While this invention has been described with respect to various specific examples and embodiments, it is to be understood that the invention is not limited thereto. For example, the triamcinolone formulations set forth herein can be used to treat conditions such as rheumatoid and osteoarthritis, as well as spinal conditions, such as facet arthritis, and treatment of chronic pain by epidural or spinal root injections of our formulations.

All references, articles, publications, patents and applications set forth above are incorporated herein by reference in their entireties.

We claim:

1. A pharmaceutical composition for treating a posterior ocular condition, the composition comprising:
(a) a triamcinolone present in a therapeutically effective amount as a plurality of particles;
(b) a viscosity inducing component in an amount effective to increase the viscosity of the composition, and;
(c) an aqueous carrier component,
wherein the composition has a viscosity of at least about 10 cps at a shear rate of about 0.1/second and is injectable into the vitreous of a human eye.

2. The pharmaceutical composition of claim 1 injectable through a 27 gauge needle into the vitreous of a human eye.

3. The pharmaceutical composition of claim 1 wherein the triamcinolone particles are substantially uniformly suspended in the composition.

4. The pharmaceutical composition of claim 1, wherein the viscosity inducing component is a polymeric hyaluronate.

5. A pharmaceutical composition for treating a posterior ocular condition, comprising:
(a) triamcinolone particles;
(b) polymeric hyaluronate, in which the triamcinolone particles are suspended;
(c) sodium chloride;
(d) sodium phosphate, and;
(e) water,
wherein the pharmaceutical composition has a viscosity at a shear rate of about 0.1/second of between about 128,000 cps and about 225,000 cps.

6. The pharmaceutical composition of claim 5, wherein the sodium phosphate present in the pharmaceutical composition comprises both monobasic sodium phosphate and dibasic sodium phosphate.

7. The pharmaceutical composition of claim 5, wherein the pharmaceutical composition comprises between about 2% w/v triamcinolone and about 8% w/v triamcinolone.

8. The pharmaceutical composition of claim 5, wherein the pharmaceutical composition comprises between about 2% w/v hyaluronate and about 3% w/v hyaluronate.

9. The pharmaceutical composition of claim 5, wherein the pharmaceutical composition comprises about 0.6% w/v sodium chloride.

10. The pharmaceutical composition of claim 5, wherein the pharmaceutical composition comprises between about 0.03% w/v sodium phosphate and about 0.04% w/v sodium phosphate.

11. A pharmaceutical composition for treating a posterior ocular condition, the pharmaceutical composition consisting essentially of:
(a) triamcinolone particles;
(b) polymeric hyaluronate, in which polymeric hyaluronate the triamcinolone particles are suspended;
(c) sodium chloride;
(d) sodium phosphate, and;
(e) water,
wherein the pharmaceutical composition has a viscosity at a shear rate 0.1/second of between about 128,000 cps and about 225,000 cps.

12. The pharmaceutical composition of claim 11, wherein the sodium phosphate present in the pharmaceutical composition is present as both monobasic sodium phosphate and dibasic sodium phosphate.

13. The pharmaceutical composition of claim 11, wherein the pharmaceutical composition has between about 2% w/v triamcinolone and about 8% w/v triamcinolone.

14. The pharmaceutical composition of claim 11, wherein the pharmaceutical composition has between about 2% w/v hyaluronate and about 3% w/v hyaluronate.

15. The pharmaceutical composition of claim 11, wherein the pharmaceutical composition has about 0.6% w/v sodium chloride.

16. The pharmaceutical composition of claim 11, wherein the pharmaceutical composition has about 0.3% w/v sodium phosphate.

17. The pharmaceutical composition of claim 11, wherein the pharmaceutical composition has between about 0.03% w/v sodium phosphate and about 0.04% w/v sodium phosphate.

18. A triamcinolone suspension for treating a posterior ocular condition, consisting of:
(a) triamcinolone particles;
(b) polymeric hyaluronate, in which the triamcinolone particles are suspended;
(c) sodium chloride;
(d) dibasic sodium phosphate heptahydrate;
(e) monobasic sodium phosphate monohydrate, and;
(f) water,
wherein the composition has a viscosity at a shear rate 0.1/second of between about 128,000 cps and about 225,000 cps.

19. The suspension of claim 18, wherein the sodium phosphate present in the suspension is present as both monobasic sodium phosphate and dibasic sodium phosphate.

20. The suspension of claim 18, wherein the suspension has between about 2% w/v triamcinolone and about 8% w/v triamcinolone.

21. The suspension of claim 18, wherein the suspension has between about 2% w/v hyaluronate and about 3% w/v hyaluronate.

22. The suspension of claim 18, wherein the suspension has between about 0.6% w/v sodium chloride.

23. The suspension of claim 18, wherein the suspension has about 0.3% w/v sodium phosphate.

24. The suspension of claim 18, wherein the suspension has between about 0.03% w/v sodium phosphate and about 0.04% w/v sodium phosphate.

25. A method for treating a posterior ocular condition, the method comprising the step of administering the pharmaceutical composition of claim 1 to the vitreous of a human or animal, thereby treating the posterior ocular condition.

26. The method of claim 26 wherein the administering step comprises intravitreal injecting.

27. A method for treating macula edema, the method comprising the step of administering to the vitreous of a human eye a pharmaceutical composition comprising:
(a) a triamcinolone, and;
(b) a hyaluronate,
wherein the pharmaceutical composition having a viscosity at a shear rate 0.1/second of between about 128,000 cps and about 225,000 cps.

28. A pharmaceutical composition for treating a posterior ocular condition, the composition comprising:
(a) a triamcinolone present in a therapeutically effective amount as a plurality of particles;
(b) a viscosity inducing component in an amount effective to increase the viscosity of the composition, and;
(c) an aqueous carrier component,
wherein the composition has a viscosity of at least about 10 cps at a shear rate of 0.1/second and is injectable into the vitreous of a human eye and wherein the pharmaceutical composition releases the triamcinolone with substantially first order release kinetics over a period of at least about 45 days after the intravitreal injection.

29. The pharmaceutical composition of claim 28, wherein the pharmaceutical composition exhibits:
(a) reduced generation of intraocular inflammation;
(b) no plume effect, and;
(c) cohesiveness, upon intravitreal injection of the pharmaceutical composition.

30. A method for treating a posterior ocular condition, the method comprising the step of intravitreal administration of a sustained release pharmaceutical composition implant comprising:
(a) a triamcinolone present in a therapeutically effective amount as a plurality of particles;
(b) a viscosity inducing component in an amount effective to increase the viscosity of the composition, and;
(c) an aqueous carrier component,
wherein the composition has a viscosity of at least about 10 cps at a shear rate of 0.1/second and is injectable into the vitreous of a human eye, and wherein the posterior ocular condition is treated for up to about 30 weeks by the triamcinolone released from the implant.

31. The method of claim 30, wherein:
(a) the pharmaceutical composition comprises triamcinolone particles, polymeric hyaluronate, in which the triamcinolone particles are suspended, sodium chloride, sodium phosphate, and water;
(b) the intravitreal administration is by injection through a 27 gauge needle into the vitreous of a human eye, and;
(c) in an aggregate number of patients practise of the method results in less intraocular inflammation that does practise of the same method with a second pharmaceutical composition which is a saline solution or suspension of a triamcinolone.

32. A process for making a pharmaceutical composition, the process comprising the steps of:
(a) mixing triamcinolone particles about 4 microns to about 8 microns in diameter with sodium chloride;
(b) heating the triamcinolone and sodium chloride mixture to a temperature between about 120° C. and about 140° C., thereby preparing a first part;
(c) mixing a sodium phosphate and water, thereby preparing a second part;
(d) dissolving sodium hyaluronate with a molecular weight between about 1.0 million Daltons and about 1.9 million Daltons in water;
(e) lyophilization of the dissolved sodium hyaluronate;
(f) reconstitution of the lyophilized sodium hyaluronate, thereby preparing a third part, and;
(g) combining the first, second and third parts, thereby making a sterile, uniform triamcinolone pharmaceutical composition which is, an opaque white gel suspension suitable for intravitreal injection to treat an ocular condition.

33. The pharmaceutical composition of claim 1 injectable through a 28 gauge needle into the vitreous of a human eye.

34. The pharmaceutical composition of claim 1 injectable through a 29 gauge needle into the vitreous of a human eye.

35. The pharmaceutical composition of claim 1 wherein the composition has a viscosity of at least about 300,000 cps at a shear rate of about 0.1/second and is injectable through a 30 gauge needle into the vitreous of a human eye.

36. A pharmaceutical composition for treating a posterior ocular condition, comprising:
(a) triamcinolone particles;
(b) polymeric hyaluronate, in which the triamcinolone particles are suspended;
(c) sodium chloride;
(d) sodium phosphate, and;
(e) water,
wherein the pharmaceutical composition has a viscosity at a shear rate of about 0.1/second of between about 80,000 cps and about 300,000 cps.

37. A pharmaceutical composition for treating a posterior ocular condition, comprising:
(a) triamcinolone particles;
(b) polymeric hyaluronate, in which the triamcinolone particles are suspended;
(c) sodium chloride;
(d) sodium phosphate, and;
(e) water, wherein the pharmaceutical composition has a viscosity at a shear rate of about 0.1/second of between about 100,000 cps and about 150,000 cps, and wherein the composition is injected into the vitreous through a 30 gauge needle.

38. The pharmaceutical composition of claim 5, wherein the pharmaceutical composition comprises between about 0.5% w/v hyaluronate and about 6% w/v hyaluronate.

39. The pharmaceutical composition of claim 5, wherein the pharmaceutical composition comprises between about 0.6% w/v sodium chloride to about 0.9% w/v sodium chloride.

40. The pharmaceutical composition of claim 5, wherein the pharmaceutical composition comprises between about 0.0% w/v sodium phosphate and about 0.1% w/v sodium phosphate.
